(19) United States
(12) Patent Application Publication
(10) Pub. No.: US 2025/0303033 A1
(43) Pub. Date: Oct. 2, 2025

(54) COMPACT WEARABLE MASSAGE AND SUCTION SYSTEM

(71) Applicant: Lilu Inc., New York, NY (US)

(72) Inventors: Sujay Suresh Kumar, New York, NY (US); Adriana Catalina Vazquez Ortiz, New York, NY (US); Ragini Kothari, New York, NY (US); Andrew Joseph Chin, New York, NY (US); Angie Y. Kim, Brooklyn, NY (US)

(21) Appl. No.: 19/091,328

(22) Filed: Mar. 26, 2025

Related U.S. Application Data
(60) Provisional application No. 63/570,861, filed on Mar. 28, 2024.

(51) Int. Cl. A61M 1/06; A61H 9/00

(52) U.S. Cl. CPC A61M 1/0697; A61H 9/0078; A61H 2201/1246; A61H 2201/1619; A61H 2201/165; A61H 2201/5005; A61H 2205/082

(57) ABSTRACT

Massaging and suction systems are provided. In an embodiment, the massaging and suction system includes a cushion device and a controller device. The cushion device includes a plurality of chambers that are interconnected by one or more channels, one or more outlets placed on the cushion device, and a flange insert. The plurality of chambers are inflated and deflated through the one or more outlets. The flange insert provides an interface for suction operations. The controller device includes a pump configured to inflate the plurality of chambers through the one or more outlets to facilitate massaging operations. In another embodiment, the massaging and suction system includes the cushion device and a controller device that includes a first pump for massaging operations and a second pump for suction operations.

### CROSS-REFERENCE TO RELATED APPLICATION

This application claims the benefit of U.S. Provisional Application No. 63/570,861, filed Mar. 28, 2024, which is incorporated by reference herein in its entirety.

### BACKGROUND

It is known that manual breast massage increases the amount of milk expressed in a breastfeeding session. Health benefits of compression massage during breastfeeding or breast pumping include preventing and relieving mastitis, plugged ducts, and engorgement. When combined with electric pumping, manual massage both increased milk production and caloric content for preterm infants. The benefit is not limited to preterm infants, however. Compression stimuli around the areola increases breast pump efficiency more generally by stimulating the release of necessary hormones. While manual massage has been used for centuries, the ability to pump efficiently with hands-free massage compression would greatly improve the modern mother's productivity.

Breast compression is also widely accepted as an effective post-surgery treatment for breast cancer related lymphedema (BCRL). One of the major important long-term complications of breast cancer treatment is lymphedema, a condition associated with adverse physical and psychosocial consequences. The major manifestation of lymphedema (LE) is chronic swelling, which causes discomfort, loss of function, and morbidity due to lymphatic impairment. Treatment of lymphedema is time consuming and expensive, and an early multidisciplinary approach is required to diagnose, treat, and prevent recurrence. Breast compression therapy has been proven to be effective in reducing swelling at the site of residual fluid, and maintaining positive postsurgical outcomes. The use of carefully selected compression garments is an effective, simple, and cheap treatment for BCRL.

Existing technologies offer systems capable of either breast pumping or massaging, often incompatible with each other or requiring complex and/or space-consuming compatibility peripherals for simultaneous breast pumping and massaging. Due to these limitations in existing technologies, there is a need for developing techniques for more compact massaging and suction systems, offering a more convenient and user-friendly experience for moms.

### SUMMARY

The present disclosure provides compact wearable systems that seamlessly integrate massaging and suction functionalities. These systems employ a streamlined approach to achieve a synchronized inflation and deflation process for massaging functions. Additionally, the systems feature a universally compatible flange or flange insert designed to accommodate a wide spectrum of suction systems.

In some examples, a compact control system is implemented for massaging functions. The system, equipped with the compact control system, may operate in conjunction with commercially available breast pump systems through the universally compatible flange or flange insert to achieve simultaneous massaging and suction functionalities. In some instances, a compact control system integrated with independent pumps may be employed, providing control over both the massaging and suction functions. The suction pump(s) may be connected to commonly used breast pump accessories/parts, such as, flange inserts, diaphragms, collector cups, one-way check valves (e.g., duckbill valves), etc., to achieve massaging and suction functionalities simultaneously. In further examples, a compact control system integrated with independent pumps may be used with customized accessories/parts to create a more compact system with combined massaging and suction functions. In some variations, the systems and/or functions disclosed herein may be implemented directly into the design of a breast pump system.

In some variations, the compact wearable systems provided herein may be utilized for diverse breast treatment or therapy applications, contributing to enhanced comfort, overall well-being, and targeted health concerns. These systems may offer benefits such as mastitis relief, lymphatic drainage, engorgement prevention, circulation enhancement, pain relief, and other advantages in various suitable scenarios.

The breast massage compression component may also be used as a stand-alone device, without a suction breast pump for various use cases, including but not limited to manual Lymphatic drainage (MLD), alleviating breast pain, Menopausal care, massaging during breastfeeding, relactation, maintenance of overall breast health, and more.

According to an aspect of the present disclosure, a massaging and suction system is provided, which includes a cushion device and a controller device. The cushion device includes a plurality of chambers that are interconnected by one or more channels, one or more outlets placed on the cushion device, and a flange insert. The plurality of chambers are inflated and deflated through the one or more outlets. The flange provides an interface for suction operations. The controller device includes a first pump configured to inflate the plurality of chambers through the one or more outlets to facilitate massaging operations.

According to an embodiment of the system, the cushion device is formed by sealing a flange layer and a cushion layer together. The flange layer includes a rim, a first opening, and the one or more outlets connectable to the controller device. The cushion layer includes a second opening, a plurality of ridges arranged along the second opening, and one or more channel-shaped structures. The first opening of the flange layer aligns with the second opening of the cushion layer. The plurality of ridges in the cushion layer and the rim of the flange layer form the plurality of chambers in the interconnected cushion. The one or more channel-shaped structures and the rim of the flange layer form the one or more channels connecting adjacent chambers of the plurality of chambers. The flange layer serves as the flange insert.

According to an embodiment of the system, the cushion device includes a flange plug having a conical shape and a cushion pad including the plurality of chambers. The one or more outlets are situated on the cushion pad. When assembled, the flange plug is inserted into a flange, and the cushion pad is placed between the flange plug and the flange. The flange plug serves as the flange insert.

According to an embodiment of the system, the cushion device further includes a flange base having a conical shape. The flange base serves as the flange, The flange base includes one or more openings. The one or more outlets on the cushion pad are connectable to the controller device through the one or more openings on the flange base.

According to an embodiment of the system, the plurality of chambers include one or more of concentric pockets, spiral configurations, paired pockets, or a series of interconnected chambers.

According to an embodiment of the system, the plurality of chambers are inflated simultaneously or sequentially.

According to an embodiment of the system, the cushion device is integrated or assembled with the flange insert. A separate suction device is configured to provide suction through the flange insert.

According to an embodiment of the system, the controller device is configured to apply a massaging operation that is synchronized with a suction operation provided by the suction device.

According to an embodiment of the system, the controller device is configured to adjust a massaging operation based on a suction operation.

According to an embodiment of the system, the system further includes one or more sensors. The controller device is configured to adjust at least one of a massaging operation or a suction operation based on data obtained from the one or more sensors.

According to an embodiment of the system, the controller device further includes a second pump configured to apply suction to facilitate suction operations.

According to an embodiment of the system, the controller device is configured to operate massage and suction functions independently.

According to an embodiment of the system, the controller device is configured to apply a massaging operation and a suction operation simultaneously.

According to an aspect of the present disclosure, a method for performing massage using a massaging and suction system is provided. The method includes inflating, by a controller device of the massaging and suction system, a plurality of chambers of a cushion device, and deflating, by the controller device of the massaging and suction system, the plurality of chambers of a cushion device. The cushion device includes the plurality of chambers that are interconnected by one or more channels, one or more outlets placed on the cushion device, and a flange insert in contact with a user. The plurality of chambers are inflated and deflated through the one or more outlets. The flange insert provides an interface for suction operations.

According to an embodiment of the method, the cushion device is formed by sealing a flange layer and a cushion layer together. The flange layer includes a rim, a first opening, and the one or more outlets connectable to the controller device. The cushion layer includes a second opening, a plurality of ridges arranged along the second opening, and one or more channel-shaped structures. The first opening of the flange layer aligns with the second opening of the cushion layer. The plurality of ridges in the cushion layer and the rim of the flange layer form the plurality of chambers in the interconnected cushion. The one or more channel-shaped structures and the rim of the flange layer form the one or more channels connecting adjacent chambers of the plurality of chambers. The flange layer serves as the flange insert.

According to an embodiment of the method, the cushion device includes a flange plug having a conical shape and a cushion pad including the plurality of chambers. The one or more outlets are situated on the cushion pad. when assembled, the flange plug is inserted into a flange, and the cushion pad is placed between the flange plug and the flange. The flange plug serves as the flange insert.

According to an embodiment of the method, the plurality of chambers are inflated simultaneously or sequentially.

According to an embodiment of the method, the method further includes applying suction to the user through the flange insert.

According to an embodiment of the method, the massaging operations and the suction are synchronized.

According to an embodiment of the method, the suction is applied using a separate suction device, or the suction is applied using the controller device of the massaging and suction system.

A machine-readable medium is provided having stored thereon a set of instructions, which if performed by one or more processors, cause the one or more processors to perform the method for performing massage using a massaging and suction system.

### BRIEF DESCRIPTION OF DRAWINGS

The present systems and methods are described in detail below with reference to the attached drawing figures, wherein:

FIG. 1 is a block diagram of a massaging and suction system implementing various functions, in accordance with some embodiments.

FIG. 2 shows an exemplary implementation of a massaging and suction system, in accordance with some embodiments.

FIGS. 3A, 3B, and 3C demonstrate various exemplary configurations that incorporate a massaging and suction system, in accordance with some embodiments.

FIGS. 4A, 4B, and 4C demonstrate another exemplary configuration of a massaging and suction system in front view, side view, and explosive view, respectively.

FIG. 5 is a flow diagram illustrating operation of an exemplary massaging and suction system, in accordance with some embodiments.

FIG. 6A is a block diagram of an exemplary network environment with a connected massaging and suction system, in accordance with some embodiments.

FIG. 6B is a block diagram of an exemplary computer system, in accordance with some embodiments.

FIG. 7 is a block diagram of an exemplary user interface (UI), in accordance with some embodiments.

FIGS. 8A, 8B, and 8C demonstrate an exemplary configuration of a massaging and suction system in explosive view, front view, and side view, respectively.

FIG. 9A provides different perspectives of a flange plug, in accordance with some embodiments.

FIG. 9B provides different perspectives of a flange base, in accordance with some embodiments.

FIGS. 9C and 9D provide different perspectives of massage cushions, in accordance with some embodiments.

FIG. 9E provides different perspectives of a diaphragm, in accordance with some embodiments.

FIG. 10A demonstrates an exemplary configuration of a massaging and suction system in explosive view.

FIG. 10B provides different perspectives of a diaphragm, in accordance with some embodiments.

FIG. 11 is a flow diagram illustrating an example process performed by a massaging and suction system, in accordance with some embodiments.

FIG. 12 demonstrates an exemplary configuration of a massaging and suction system in explosive view.

FIG. 13 demonstrates another exemplary configuration of a massaging and suction system, in accordance with some embodiments.

FIGS. 14A-14C demonstrate exemplary variants of the massage cushions, in accordance with some embodiments.

FIGS. 15A-15D illustrates examples of UIs, in accordance with some embodiments.

### DETAILED DESCRIPTION

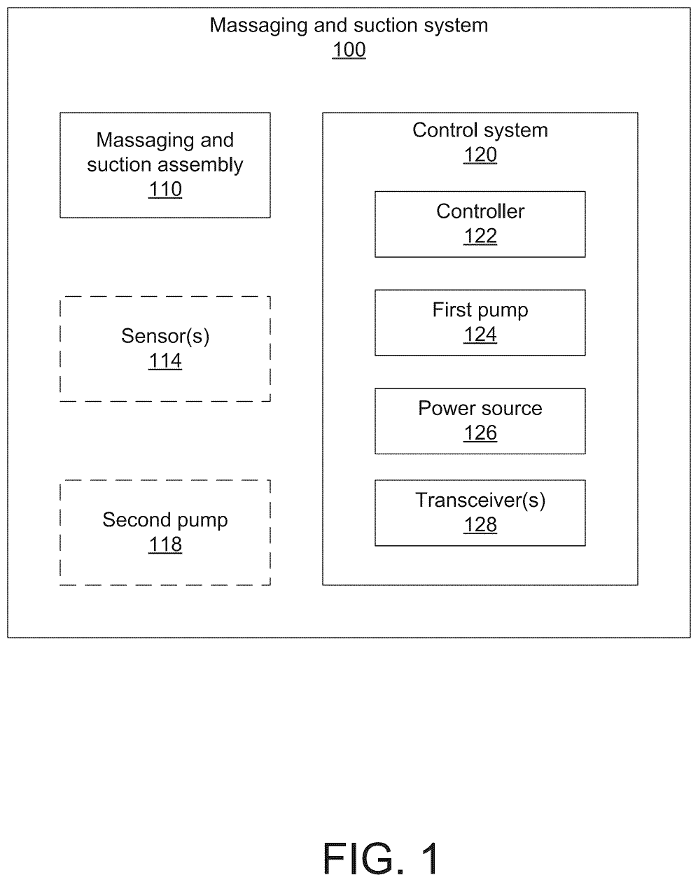

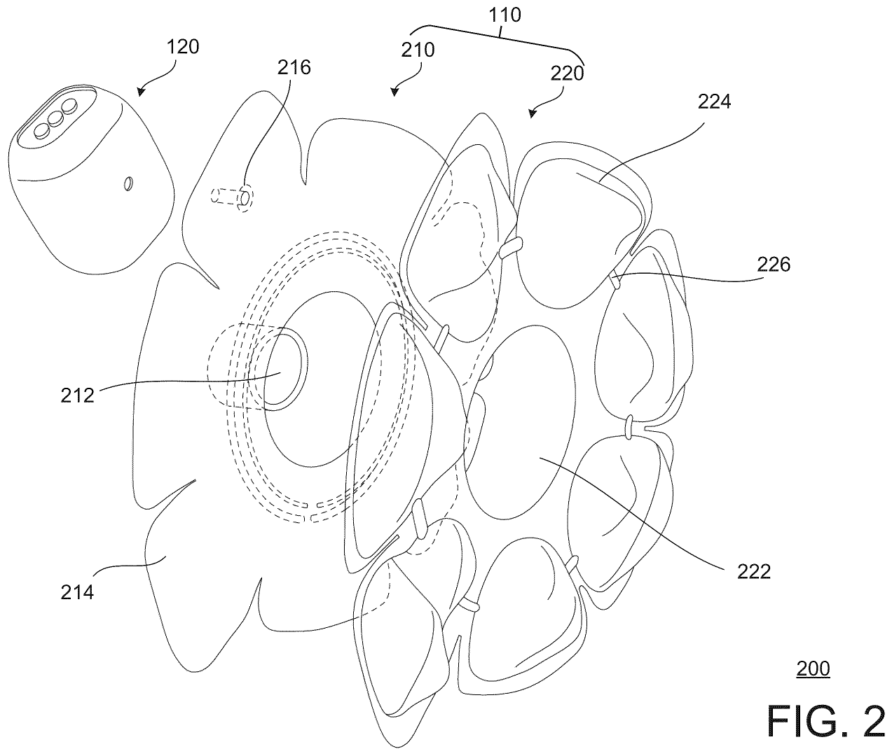

FIG. 1 is a block diagram of a massaging and suction system 100 implementing various functions, in accordance with some embodiments. The massaging and suction system 100 is designed to be a compact system that can be used with various bras or undergarments. For example, users can place the massaging and suction system 100, such as the massaging and suction assembly (or attachment) 110, inside the cup of any bra/garment, allowing the system 100 to adhere to the breast, with the bra/garment providing support for the system during use. The massaging and suction system 100 may work alongside or in conjunction with other breast pumps or suitable suction devices. The system 100 may operate on one or both breasts of the user simultaneously. Various example implementations of the massaging and suction system 100, along with variations of its components, will be described with reference to FIGS. 2-15 hereinafter. However, it should be noted that these examples are provided to illustrate the principles of the invention and do not limit its scope. For example, other suitable components, designs, and arrangements may also be used to facilitate the functions described herein. In certain embodiments, the massaging and suction system 100 provides a versatile and adaptive solution, integrating massage and suction into a compact, intelligent, and user-friendly device. In certain embodiments, the massaging and suction system 100 offers cross-compatibility with various breast pump systems, serving as an accessible and effective lactation support system for diverse user needs.

FIG. 2 shows an exemplary implementation 200 of the massaging and suction assembly 110. As shown in FIG. 1 and FIG. 2, the massaging and suction system 100 may include various components, such as a massaging and suction assembly 110 and a control system 120.

The massaging and suction assembly 110 includes a plurality of interconnected cushions designed to provide massage to the breast (or both breasts independently or simultaneously). The massaging and suction assembly 110 may be powered by a pneumatic or hydraulic mechanism to perform breast massage. The cushions are referred to as bladders or chambers hereinafter. As shown in FIG. 2, the massaging and suction assembly 110 is formed by a flange layer 210 and a cushion layer 220. The flange layer 210 includes a tunneled opening 212 that extends in a specific direction to form a frustum shape. The opening 212 may be centrally positioned or arranged in other suitable ways. The opening 212 may be inserted or attached into a flange connected to a breast pump, allowing the breast pump to apply suction around the nipple. Additionally, the flange layer 210 may include an outer edge (or rim) 214 extending from the bottom of the tunneled opening 212. The outer edge (or rim) 214 may be shaped to be relatively wider and flatter, conforming to the natural contour of the breast. The flange layer 210 further includes an outlet 216 disposed on the outer edge (or rim) 214. The outlet 216 may connect to a pump (e.g., in device 120) either directly (e.g., through insertion) or via a tube/hose. The outlet 216 is situated on one side of the flange layer 210, while the other side of the flange layer 210 forms the plurality of interconnected bladders when sealed with the cushion layer 220. An outlet 216 may be any type of port, such as a nozzle, which includes an opening that allows air or fluid to pass through. In some instances, an outlet 216 may include a valve that regulate or stop the flow of fluid or air.

In certain embodiments, the flange layer 210 may serve as a flange insert. A flange insert refers to a removable component that fits inside a flange, such as a breast pump, to provide a better seal, improve comfort, and/or adjust the flange size for a better fit around the nipple. In some examples, either a single flange layer or multiple layers of flange inserts may be used simultaneously. The flange layer 210 may serve as one of the insert layers during use.

The cushion layer 220 has an opening 222 that aligns with the opening 212 of the flange layer 210 when the two layers are sealed together. The cushion layer 220 is designed with a plurality of malleable ridges (e.g., 224) arranged along the opening 222. Additionally, the cushion layer 220 incorporates a channel-shaped design between each pair of adjacent ridges. When the flange layer 210 and the cushion layer 220 are sealed together, most of the lower periphery of each ridge binds with the flange layer 210, forming a respective bladder. The channel-shaped design between each pair of adjacent ridges binds with the flange layer 210, forming a respective channel (e.g., 226) and facilitating air/fluid connectivity between the corresponding adjacent bladders. This way, the plurality of bladders (e.g., 224) in the massaging and suction assembly 110 are linked through small channels (e.g., 226), allowing for inflation and deflation of all bladders through the outlet 216. In some examples, the plurality of bladders may be sequentially inflated and/or deflated as air or liquid flows into or out of the bladders through the outlet 216. In some instances, multiple outlets 216 may be distributed along the flange layer 210, enabling air/fluid flow into or out of multiple bladders simultaneously. This configuration can achieve a constant and uniform compression around the entire breast.

In some examples, the ridges may be made of any suitable elastic materials such as silicone, rubber, or flexible plastic, and may be formed using any suitable process, such as thermal process, welding process or the like.

In some instances, as depicted in FIG. 2, the edge of the cushion layer 220 may be designed with contours matching the shape of the plurality of bladders (or ridges), and correspondingly, the outer edge (or rim) 214 of the flange layer 210 may adopt a similar design.

The side of cushion layer 220 of the massaging and suction assembly 110 faces the breast when the massaging and suction system 100 or assembly 110 is worn by a user. The plurality of bladders in the massaging and suction assembly 110 may simulate fingertip-like pressure and produce lactation massage effects as the bladders are inflated and deflated.

Referring back to FIG. 1, the control system 120 is configured to control the operation of components in the massaging and suction system 100, such as the massaging and suction assembly 110. The control system 120 includes a controller 122, a first pump 124, a power source 126, and one or more transceivers 128. In some variations, the control system 120 may be connected to multiple massaging and suction systems, and each massaging and suction system may be embodied as the massaging and suction system 100 described in the present disclosure.

The controller 122 is configured to generate instructions and/or control signals for controlling one or more components in the massaging and suction system 100. The controller 122 may encompass a microcontroller, control circuits, semiconductor chips/modules, a general-purpose or special-purpose processor, memory, or any combination thereof to facilitate the generation of instructions and/or control signals.

The first pump 124 is connectable to the massaging and suction assembly 110 and is configured to operate with a solenoid valve to deliver air/fluid to inflate/deflate the plurality of bladders in the massaging and suction assembly 110. It will be understood by one skilled in the art that other suitable valves may be utilized for distribution of air/fluid. The first pump 124 is communicatively connected to the controller 122 and is configured to operate in response to the instructions and/or control signals from the controller 122. In some variations, the controller 122 may generate various instructions and/or control signals to enable the first pump 124 to operate in different modes. Each mode may be defined by a set of parameters, such as the cycle, frequency, and intensity of air/fluid suction or delivery. These modes may influence the way the plurality of bladders inflate/deflate.

As shown in FIG. 2, air/fluid passes through a single outlet (e.g., 216) from the control system 120, distributing to all interconnected bladders. This configuration may facilitate a synchronized inflation and deflation process, applying consistent pressure across the breast. In an example, the first pump may be an air pump that operations in conjunction with a solenoid valve (not shown in FIG. 1) within the control system 120 to drive massage effects through the interconnected bladders with the inner surface (e.g., the surface of the cushion layer 220) featuring a plurality of ridges.

The power source 126 is configured to provide power to one or more components in the massaging and suction system 100, including the controller 122, the first pump 124, the transceiver(s) 128, and/or other suitable devices/systems in the massaging and suction system 100.

The transceiver(s) 128 is configured to transmit/receive data from internal components and/or external devices/systems.

The control system 120, including the controller 122, the first pump 124, the power source 126, the transceiver(s) 128, and other suitable circuitry or hardware components (e.g., one or more solenoid valves), may be integrated within a housing. The housing may include buttons or a touch-screen for user inputs. Additionally and/or alternatively, the control system 120 may communicate with a remote controller to receive user inputs.

In some instances, the massaging and suction system 100 may further include one or more sensors 114. The one or more sensors 114 may be situated on or integrated into the massaging and suction assembly 110 and/or the control system 120 to monitor the operation of one or more components in the massaging and suction system 100 and/or to track breast milk production. For example, various sensors, such as flow sensors, vacuum sensors, or pressure sensors, may be employed to monitor the performance of the system 100 (e.g., the performance of one or more pumps). Other sensors, such as infrared sensors, sonic sensors, flow rate detection sensors, or conductance/resistance-based sensors, may be utilized to measure changes in breast volume, track milk production, and/or monitor other biomarkers of the user.

In some examples, the massaging and suction system 100 may include various sensors and/or devices for monitoring its overall functioning. The following discusses several sensors as examples.

Vacuum Gauge Sensors: these sensors may be used to monitor and control the suction pressure applied to mimic the baby's natural sucking pattern. Adjustable pressure settings can accommodate individual comfort levels.

Air Pressure Sensors: these sensors may be used to monitor and adjust the intensity of the massage compression.

System Temperature Sensors: these sensors may be used to monitor that components within the device are within safe temperature ranges.

Battery Level Sensors: these sensors may be used to monitor battery levels. Wearable breast pumps are typically powered by rechargeable batteries. Including a sensor to monitor the battery level helps users plan charging sessions and ensures the pump is ready for use when needed.

Bluetooth or Wi-Fi Connectivity devices. These devices may be implemented to enable connectivity features. Connectivity features enable the breast pump to sync with a smartphone app. This allows users to track pumping sessions, set reminders, and receive personalized tips or information.

Positioning Sensors: these sensors are used to detect the proper placement of the breast pump, ensuring optimal performance and comfort during use.

Smart Controls Sensors (or Devices): these sensors/devices may include capacitive touch sensors or gesture recognition sensors, which may be utilized for easy and discreet control of the breast pump without physical buttons.

Milk Detection Sensors: these sensors may include optical sensors configured to detect the presence of milk in the pumping mechanism, preventing overflows and/or ensuring efficient milk expression.

Noise Level Sensors: these sensors are used to monitor noise levels, which may be important for those who value discretion during pumping. This helps design a quieter pump for more discreet use.

Skin Contact Sensors: these sensors are used to detect skin contact and configured to initiate or pause pumping sessions when the breast pump is properly positioned.

In some examples, the massaging and suction system 100 may include various sensors and/or devices for monitoring general breast health, breast milk output and conditions such as lymphedema, breast cancer, breast pain from hormonal conditions or diseases. The following discusses several sensors as examples.

Temperature Sensors may be used to monitor the temperature of the breast during and after pumping sessions could be useful. Persistent changes in temperature could potentially be indicative of inflammation or other issues. Additionally differential changes in temperature have been shown to be correlated with early signs of breast cancer.

Flow sensors may be used to measure the volume of milk expressed per unit of time. This information can help track pumping sessions and provide insights into milk production.

Infrared Imaging or Thermography sensors/devices may be utilized. Advanced breast pumps could integrate infrared sensors or thermographic cameras to capture temperature variations across the breast surface. Abnormal heat patterns might signal potential issues.

A sensor that assesses breast density may be incorporated to provide information about the composition of breast tissue (e.g., for breast density measurement). Dense breast tissue is associated with a higher risk of breast cancer.

Stretch and/or pressure sensors may be utilized to measure changes in breast size/volume. Sensors that measure changes in the size or shape of the breast could potentially help in detecting early signs of lymphedema, a condition that may occur after breast cancer treatment. Change in breast size can also be used to estimate breast milk production and expression.

Biometric sensors may be used to monitor physiological parameters, such as heart rate or respiratory rate during pumping sessions might offer additional insights into overall health and stress levels.

Acoustic sensors may be used to analyze sounds produced during pumping sessions. Unusual sounds or patterns might be indicative of changes in breast tissue or error in breast pump mechanism that could warrant further investigation.

Electrical Impedance Imaging sensors/devices may be used to measure electrical conductivity of tissues, providing information about tissue composition. Changes in impedance could be associated with certain breast conditions.

PH sensors may be used to monitor the pH levels of expressed milk or the breast tissue itself might offer information about potential infections or other abnormalities.

It will be recognized that other types of sensors may be integrated into the massaging and suction system 100, controlled by the controller 122, and configured to monitor various suitable aspects/parameters of the massaging and suction system 100 during operation.

In some examples, the massaging and suction system 100 may further include a second pump 118 configured to apply suction to the nipple for breast pumping. The second pump 118, along with other components (e.g., a corresponding solenoid valve, associated circuitry, etc.), may be part of a device/system independent of the control system 120. In this arrangement, the independent control systems may be housed separately. Alternatively, the second pump 118 may be integrated in the control system 120, controlled by the controller 122, and powered by the power source 126. In this configuration, the control system 120 functions as an integrated system, housing all components within a single enclosure. The second pump 118 may include a suction pump and a corresponding solenoid valve, operating collaboratively to apply various suction patterns on the breast through the universally compatible flange within the massaging and suction assembly 110. In certain embodiments, as an integrated system, the control system 120 may provide the massage functions and the suctions functions independently.

In some instances, the massaging and suction system 100 may be used as a stand-alone device, without a suction breast pump (e.g., the second pump 118) for various use cases, including but not limited to Manual Lymphatic Drainage (MLD), alleviating breast pain, Menopausal care, massaging during breastfeeding, relactation, maintenance of overall breast health, and more.

MLD is a type of gentle massage technique that may be used to manage lymphedema. This involves specific, light-touch massage movements that aim to encourage the movement of lymphatic fluid and reduce swelling. To facilitate MLD, the massaging and suction assembly 110 in the massaging and suction system 100 may be controlled to perform gentle compression on the affected breast. Such breast compression may also be part of managing lymphedema. The breast compression may be customized based on consulting with a lymphedema therapist or healthcare provider to ensure that the techniques are appropriate and safe for an individual's condition.

Breast pain may be related to various conditions, such as Fibrocystic Breast Condition. For conditions like fibrocystic breasts, where lumps or pain may be present, gentle breast massage or compression may offer some relief, which may be provided by the massaging and suction system 100 provided herein. It's essential to be cautious and gentle to avoid exacerbating any discomfort.

The massaging and suction system 100 may be utilized to assist with Menopausal care that involves medical and supportive measures taken to manage and alleviate symptoms associated with menopause. Regular breast self-massage or examination may be employed as part of breast health awareness during menopause. Women in menopause may also experience changes in breast tissue density and sensitivity. The massaging and suction system 100 may be utilized to help detect any changes or abnormalities early on.

The massaging and suction system 100 may be utilized for gentle massage during breastfeeding to support milk flow and empty breasts fully. Furthermore, the massaging and suction system 100 may be utilized for relactation that is the process of reestablishing breastfeeding after a period of not breastfeeding or lactating.

Additionally, the massaging and suction system 100 may be utilized for maintaining overall breast health through breast massage for hormonal conditions such as Polycystic Ovary Syndrome (PCOS) and endometriosis among others.

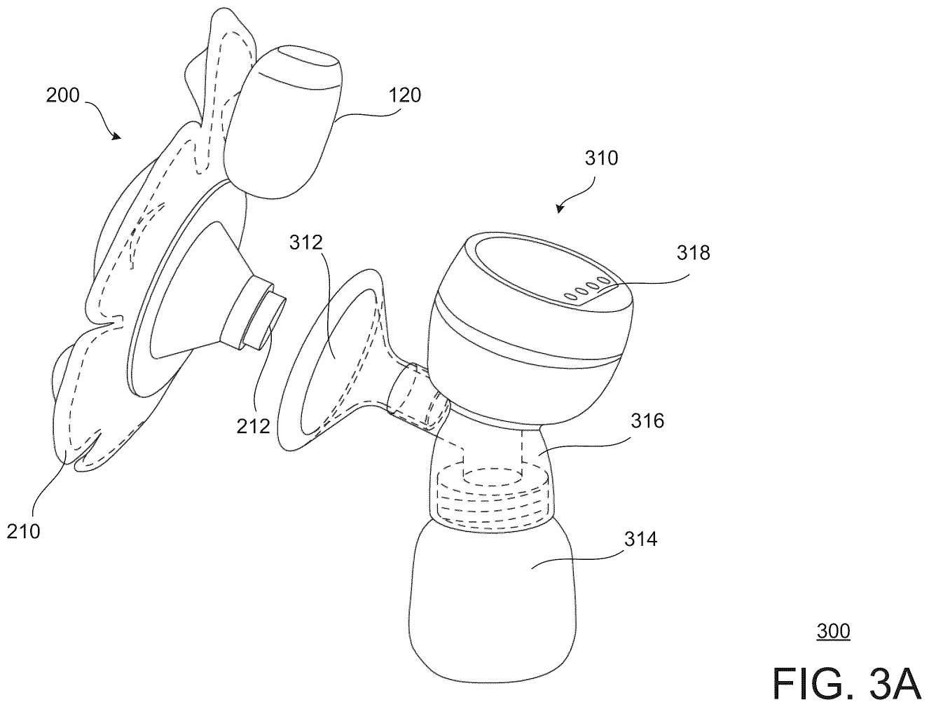

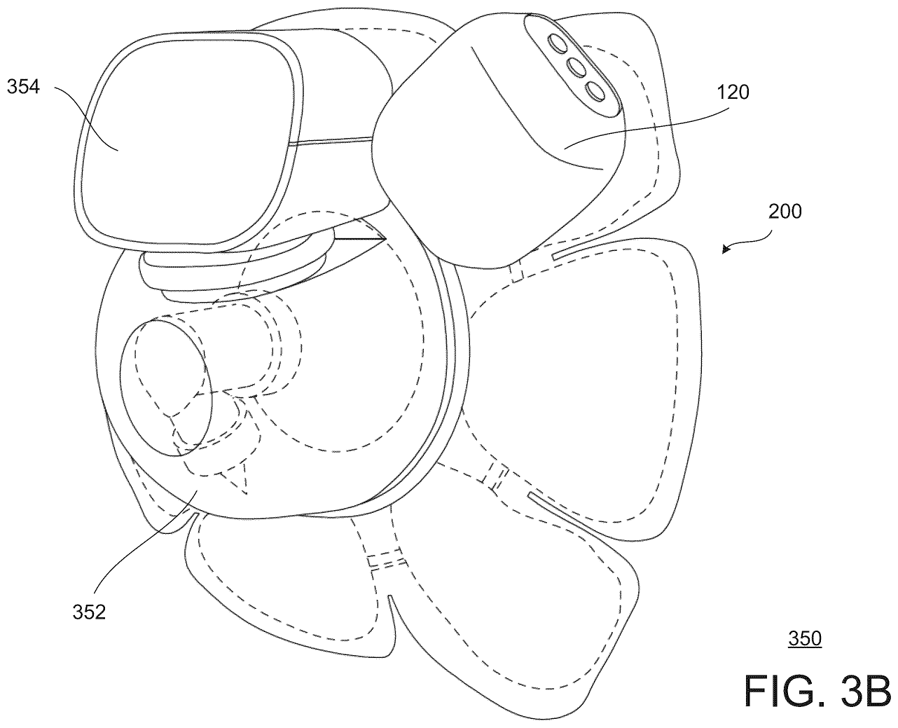

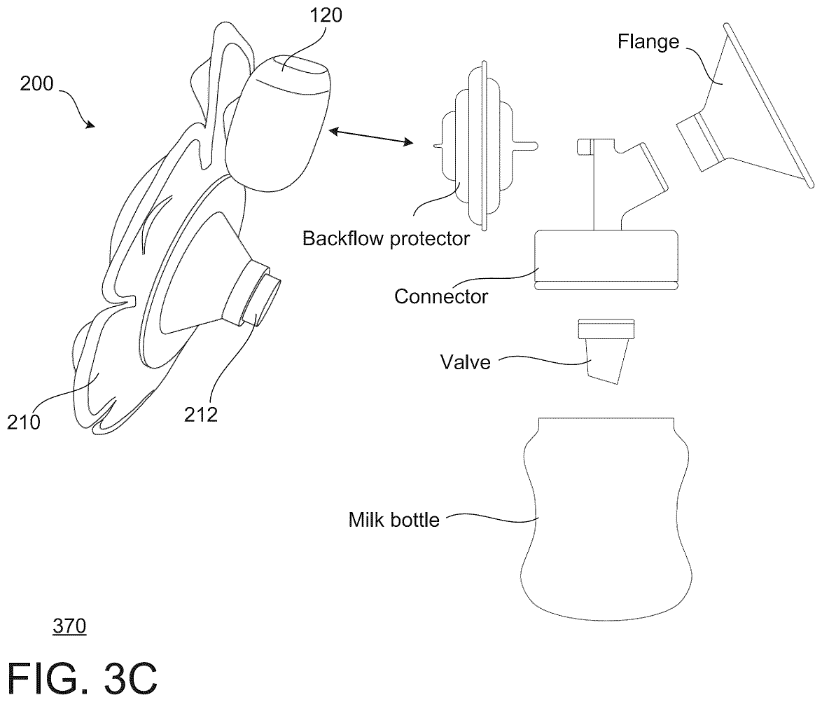

FIGS. 3A and 3B illustrate various exemplary configurations incorporating the massaging and suction system 200 as illustrated in FIG. 2, in accordance with some embodiments.

In FIG. 3A, the massaging and suction system 200 is connected to an independent breast pump system 310 via a flange 312. The flange 312 may be connected to a milk bottle 314 through a connector 316 coupled to a one-way check valve (such as a duckbill valve, not shown in FIG. 3A).

In this example, the breast pump system 310 is controlled by a separate control system 318, facilitating breast pumping operations. During operation, the opening 212 of the flange layer 210 within the massaging and suction system 200 is inserted or attached to the breast pump system 310 through the flange 312, allowing the breast pump system 310 to apply suction around the nipple through the opening 212. The extracted milk will flow through the connector 316 and the one-way check valve, eventually being collected by the milk bottle 314. Additionally, the control system 120 may operate to control the massaging and suction system 200, enabling simultaneous performance of massaging and suction operations. The flange layer 210 with the opening 212 is designed to be universally compatible with commonly used breast pump accessories/parts, such as the flange 312, allowing the system 200 to be compatible with a variety of existing devices/systems available in the market. It will be recognized that the breast pump system 310 may include an electric pump or a manual pump.

FIG. 3B demonstrates another exemplary configuration 350 of a more compact design. In this example, a collector cup 352 is designed to cover the opening 212 of the flange layer 210 within the massaging and suction system 200. Additional parts, such as a connector, a one-way check valve (e.g., a duckbill valve), and/or a diaphragm may be assembled between the opening 212 and the collector cup 352. This configuration allows for the application of suction to the nipple through the opening 212 during breast pumping and facilitates milk collection. The collector cup 352 includes a first outlet connectable to a pump system 354 that operates to provide suction. In some variations, the collector cup 352 may include a second outlet connectable to another milk bottle for milk collection. Additionally, the control system 120 may operate to control the massaging and suction system 200, enabling simultaneous performance of massaging and suction operations.

FIG. 3C demonstrates another exemplary configuration 370. In this configuration 370, the suction pump, along with its associated circuitry and/or hardware components (e.g., some or all of the components in the control system 318 or 354), may be integrated in the control system 120 within the massaging and suction system 200. In this setup, the suction pump within the control system 120 may be connected to the breast pump parts, such as an outlet located on a backflow protector, using an appropriate connection method 380, such as tubing. This way, the control system 120 may operate to control the massaging and suction system 200, enabling simultaneous performance of massaging and suction operations.

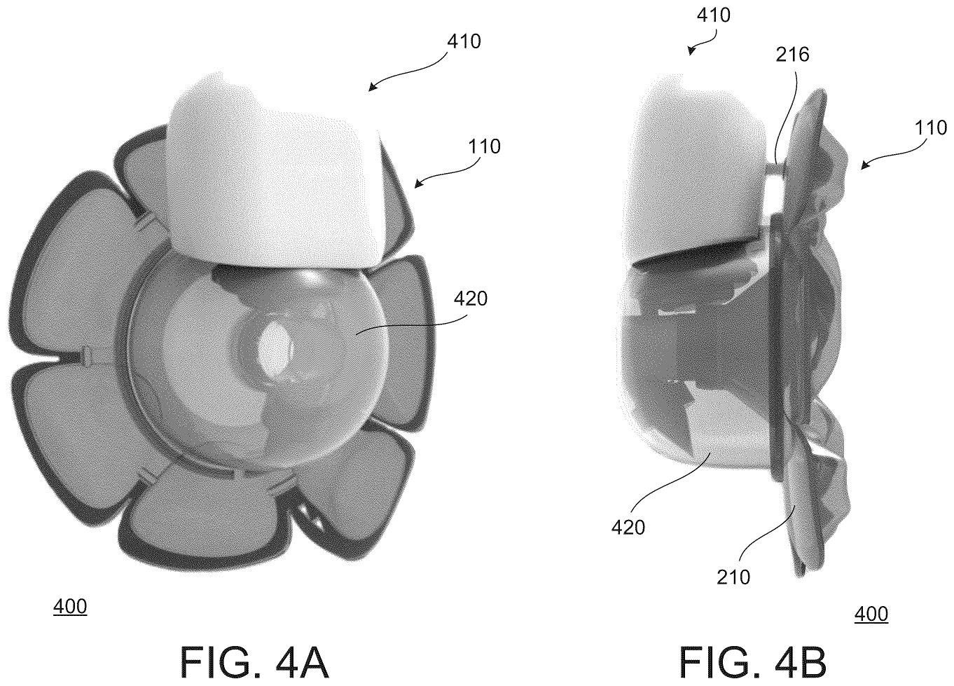

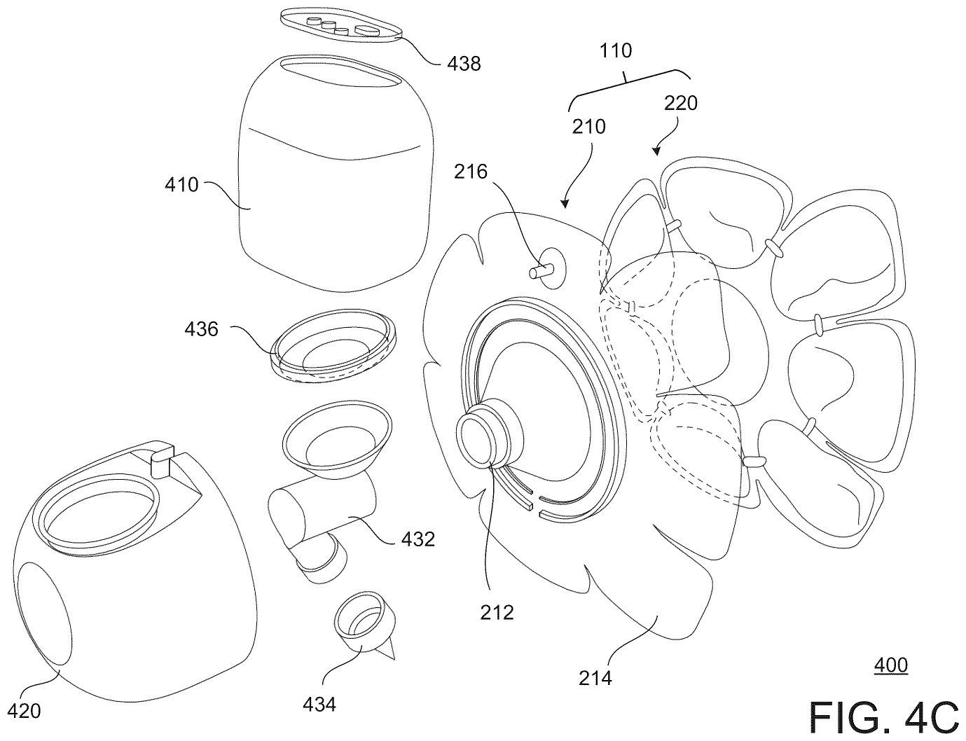

FIGS. 4A and 4B demonstrate an exemplary configuration 400 of a massaging and suction system 100 in front view and side view, respectively.

In this configuration 400, the massaging and suction system 100 is formed by a massaging and suction assembly 110 and a control system 410. The control system 410 may be implemented as the control system 120 integrated with the second pump 118 as shown in FIG. 1. The control system 410 is connectable to the massaging and suction assembly 110 via the outlet 216 positioned on the flange layer 210. A milk collection part 420 is designed to cover at least a portion of the flange layer 210. The control system 410 is attachable to the milk collection part 420 and connectable to a connector (e.g., the connector 432 as shown in FIG. 4C) through an opening situated on the milk collection part 420. The control system 410 may control the inflation/deflation of the massaging and suction assembly 110 using a first pump and a corresponding valve (e.g., a solenoid valve) and control the suction process using a second pump and a corresponding valve (e.g., a solenoid valve) within the control system 410. The solenoid valves may be integrated within the control system 410.

FIG. 4C is an explosive view of the exemplary configuration 400 as depicted in FIGS. 4A and 4B. As shown in FIG. 4C, the massaging and suction assembly 110 includes the flange layer 210 and the cushion layer 220. The plurality of bladders in the massaging and suction assembly 110 are inflated or deflated through the outlet 216 located on the rim 214 of the flange layer 210. During assembly, the opening 212 may be inserted into a connector 432 through a side opening. The connector 432 may further include a first opening and a second opening on opposite ends. The first opening may be connected to the control system 410 through an adaptor/connector part 436. The second opening of the connector 432 may be connected to a valve 434 (e.g., a one-way check valve) to direct the extracted milk for collection by the milk collection part 420. The milk collection part 420 encloses all these components and provides a sealed environment to guide suction applied to the nipple and channel the extracted milk into the part 420.

The control system 410 includes the first pump 124, along with its associated circuitry and/or hardware components, configured for massaging operations, and the second pump 118, along with its associated circuitry and/or hardware components, configured for suction operations. The housing of the control system 410 further includes a panel 438, which may include one or more buttons and/or other types of user interfaces to enable user inputs.

The first pump may be an air or fluid pump, while the second pump may be a vacuum/suction pump. The first pump operates in conjunction with the corresponding solenoid valve to pass air through the outlet 216, distributing the air to all interconnected bladders in the massaging and suction assembly 110. This way, a synchronized inflation and deflation process may be facilitated, ensuring consistent pressure across the breast. The second pump 118 operates in conjunction with the corresponding solenoid valve to apply various suction patterns on the breast through the universally compatible flange within the massaging and suction assembly 110. The suction patterns may be optimized to ensure efficient and comfortable milk expression for users as well as offer users the option to toggle between the levels/intensity of suction so they can utilize settings that work best for them. Expressed breast milk may be channeled and collected into a collector part which may be attached to or integrated in the massaging and suction system of the configuration 400.

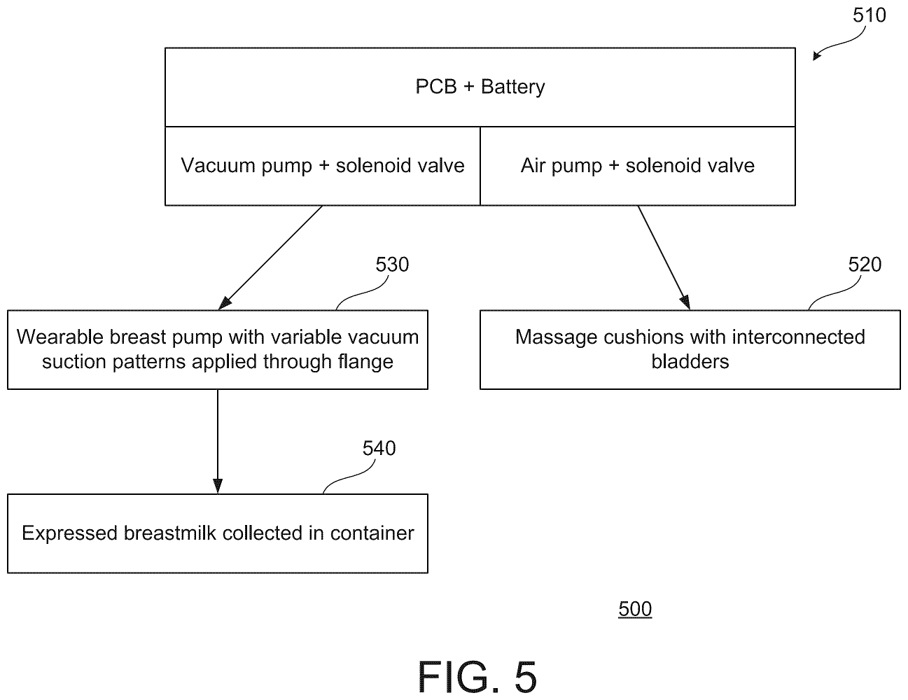

FIG. 5 is a flow diagram illustrating operation of an exemplary massaging and suction system 500, in accordance with some embodiments. In this example, the massaging and suction system 500 implements the configuration 400 as shown in FIGS. 4A, 4B, and 4C. However, it should be noted that the massaging and suction system 500 may also implement other configurations described in the present application and/or incorporate other suitable variations.

In FIG. 5, a control system 510 includes a printed circuit board (PCB) integrated with control circuits, chips, and/or modules to facilitate various control functions, and a battery device/module to power the control system 500. In this example, the control system 510 is integrated with an air (or fluid) pump with a corresponding solenoid valve associated with massage functions, and a vacuum (or suction) pump with a corresponding solenoid valve associated with suction functions.

During operation, the massaging and suction system 500 may be assembled in a manner similar to the arrangement shown in FIGS. 4A, 4B, and 4C, with the appropriate accessories/parts, and then placed on the user's breast.

In block 520, the air pump with the corresponding solenoid valve is connected to massage cushions with interconnected bladders (e.g., the massaging and suction assembly 110) through a single outlet (e.g., 216).

In block 530, the vacuum pump with the corresponding solenoid valve functions as a wearable breast pump with variable vacuum suction patterns applied to the nipple through a flange (e.g., connected to the flange layer 212 as shown in FIG. 2). In block 540, the expressed milk may be collected in a collector (e.g., the milk collection part 420 as shown in FIG. 4B or 4C, or the collector cup 352 as shown in FIG. 3B).

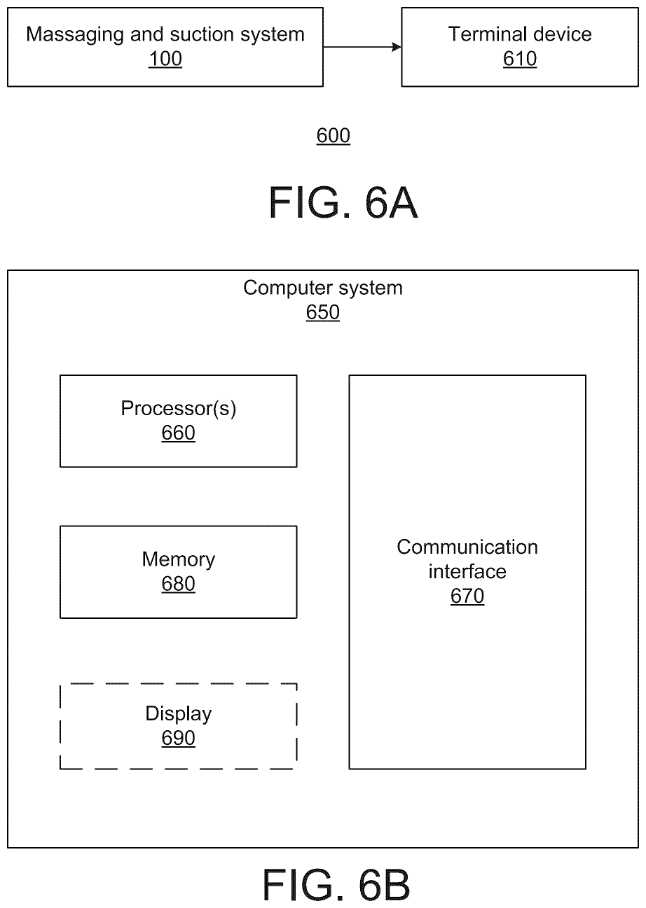

FIG. 6A is a block diagram of an exemplary network environment 600 with a connected massaging and suction system, in accordance with some embodiments.

As shown in FIG. 6A, the network environment 600 may include various components, such as the massaging and suction system 100 as depicted in FIG. 1 and a terminal device 610.

The terminal device 610 may include at least some of the components, features, and functionality of the example computer system 650 of FIG. 6B. By way of example and not limitation, a terminal device 610 may be embodied as a Personal Computer (“PC”), a laptop computer, a mobile device, a smartphone, a tablet computer, a virtual reality headset, or any other suitable device.

The massaging and suction system 100 may be connected to the terminal device 610 through wired and/or wireless networks. For example, the wired connection may include ethernet cable, USB cable, and etc. The wireless connection may include WiFi, Bluetooth, cellular network (e.g., the second, third, fourth, and/or fifth generation), near field communication (NFC), and etc.

The terminal device 610 may provide a user interface that enables users to monitor the operation of the massaging and suction system 100 and/or input commands to control the operation of the massaging and suction system 100.

FIG. 6B is a block diagram of an exemplary computer system 650 configured to implement various functions, in accordance with some embodiments. In some examples, the computer system 650 may be implemented in the massaging and suction system 100 and/or the terminal device 610 in the network environment 600 as shown in FIG. 6B.

As shown in FIG. 6B, the computer system 650 may include one or more processors 660, a communication interface 670, a memory 680, and a display 690. The processor(s) 660 may be configured to perform the operations in accordance with the instructions stored in the memory 680. The processor(s) 660 may include any appropriate type of general-purpose or special-purpose microprocessor (e.g., a CPU or GPU, respectively), digital signal processor, microcontroller, or the like. The memory 680 may be configured to store computer-readable instructions that, when executed by the processor(s) 660, can cause the processor(s) 660 to perform various operations disclosed herein. The memory 680 may be any non-transitory type of mass storage, such as volatile or non-volatile, magnetic, semiconductor-based, tape-based, optical, removable, non-removable, or other type of storage device or tangible computer-readable medium including, but not limited to, a read-only memory (“ROM”), a flash memory, a dynamic random-access memory (“RAM”), and/or a static RAM. Various processes/flowcharts described in terms of mathematics in the present disclosure may be realized in instructions stored in the memory 680, when executed by the processor(s) 660.

The communication interface 670 may be configured to communicate information between the computer system 650 and other devices or systems, such as the compression system 100 and/or the terminal device 610 as shown in FIG. 6B, or other suitable devices/systems connected to the terminal device 610. For example, the communication interface 670 may include an integrated services digital network (“ISDN”) card, a cable modem, a satellite modem, or a modem to provide a data communication connection. As another example, the communication interface 670 may include a local area network (“LAN”) card to provide a data communication connection to a compatible LAN. As a further example, the communication interface 670 may include a high-speed network adapter such as a fiber optic network adaptor, 10G Ethernet adaptor, or the like. Wireless links can also be implemented by the communication interface 670. In such an implementation, the communication interface 670 can send and receive electrical, electromagnetic or optical signals that carry digital data streams representing various types of information via a network. The network can typically include a cellular communication network, a Wireless Local Area Network (“WLAN”), a Wide Area Network (“WAN”), or the like.

The communication interface 670 may also include various I/O devices such as a keyboard, a mouse, a touchpad, a touch screen, a microphone, a camera, a biosensor, etc. A user may input data to the computer system 650 (e.g., a terminal device) through the communication interface 670.

The display 690 may be integrated as part of the computer system 650 or may be provided as a separate device communicatively coupled to the computer system 650. The display 190 may include a display device such as a liquid crystal display (“LCD”), a light emitting diode display (“LED”), a plasma display, or any other type of display, and provide a graphical user interface (“GUI”) presented on the display for user input and data depiction. In some embodiments, display 690 may be integrated as part of the communication interface 670.

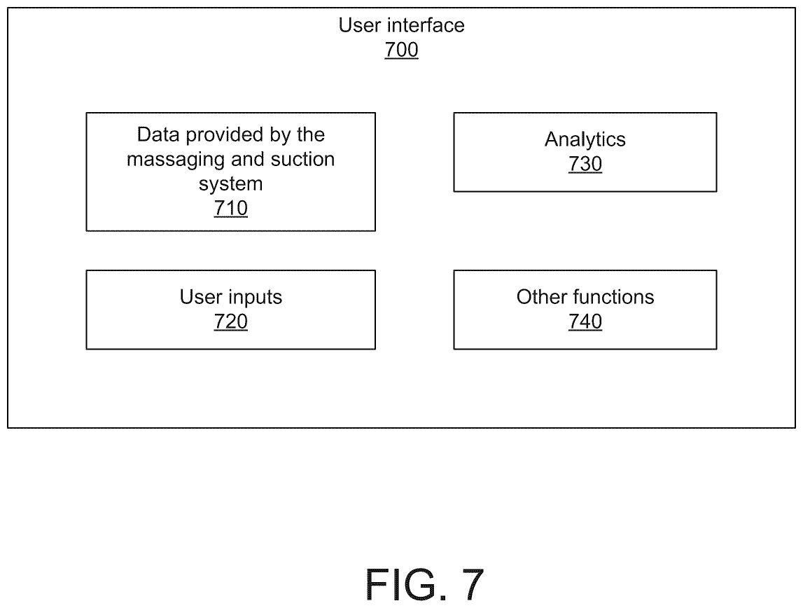

FIG. 7 is a block diagram of an exemplary user interface (UI) 700, in accordance with some embodiments. The blocks as depicted in FIG. 7 shows exemplary functions provided by a terminal device 600, to facilitate interactions with a massaging and suction system 100 as shown in FIG. 6A.

As shown in FIG. 7, the terminal device 600 may provide functions 710 related to processing data provided by the massaging and suction system 100, functions 720 related to interactions with a user, functions 730 for generating analytics (e.g., graphical or textual), and other functions 740. The terminal device 600 may communicate with the massaging and suction system 100 via a wired or wireless network. The terminal device 600 may display data (e.g., the analytics from block 730) and/or receive user inputs (e.g., from block 720) via the communication interface 670.

In some examples, the terminal device 600 may perform the functions 710 to process the data received from the massaging and suction system 100 to obtain information associated with massaging and/or breast pumping, and execute the functions 730 to display the generated analytics information in the UI. The information may include a quantity of milk produced/stored in breasts, a quantity of milk expressed, changes in milk volume production over short intervals (e.g., during an event of breastfeeding/pumping) or long intervals (e.g., days, weeks, months, etc.), rate of milk production over short/long intervals, irregularities in milk production/output, irregularities in duration of breastfeeding or pumping, irregularities in schedule of breastfeeding or pumping, optimal suction and massage patterns, settings for individual users, and/or any other suitable information.

In some variations, the terminal device 600 may perform the functions 720 to receive user inputs via I/O devices (e.g., in the communication interface 670). The terminal device 600 may cause display of preferences, which could include default values or inputs by the user, in the UI. Examples of the user inputs/preferences may include a daily diet, a baby's sleeping schedule, a baby's weight, changes in a baby's weight, the user's sleeping schedule, illness of the baby/user, the user's daily work schedule, the user's mental health status, and any other suitable information. Additionally, the terminal device 600 may determine default values for certain preferences based on patterns discovered from sensor data provided by the compression system 100. The terminal device 600 may determine measurement settings (e.g., selection of node pairs) based on the user inputs. The user may be able to manually select the node pair(s) and/or measurement settings via options provided by the terminal device 600.

The terminal device 600 may provide other functions 740 to the user. The other functions 740 may involve an action to be performed by the terminal device 600 or the massaging and suction system 100. Additionally, the other functions 740 may enable the user to link to online resources (e.g., medical database, healthcare network, social media platforms) to obtain additional information.

In certain embodiments, unlike the integral configuration illustrated in FIGS. 2-4, the massaging and suction assembly 110 may include a plurality of discrete components, which can be assembled to form an assembly/attachment capable of integrating massage functions into a massaging and suction assembly 110 that supports both massage and suction functions.

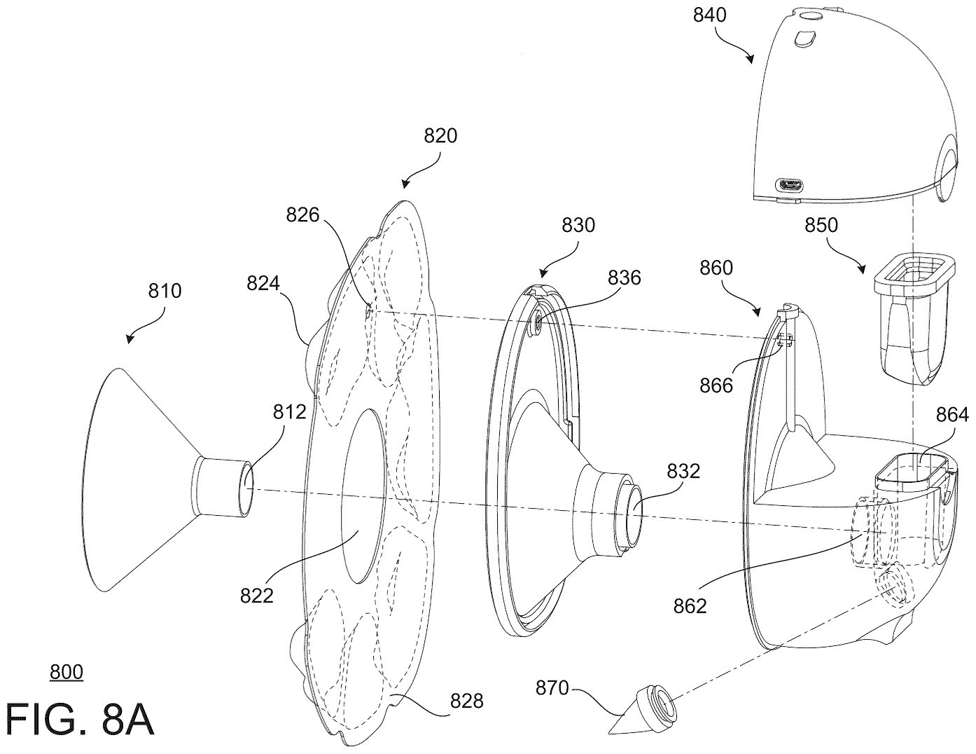

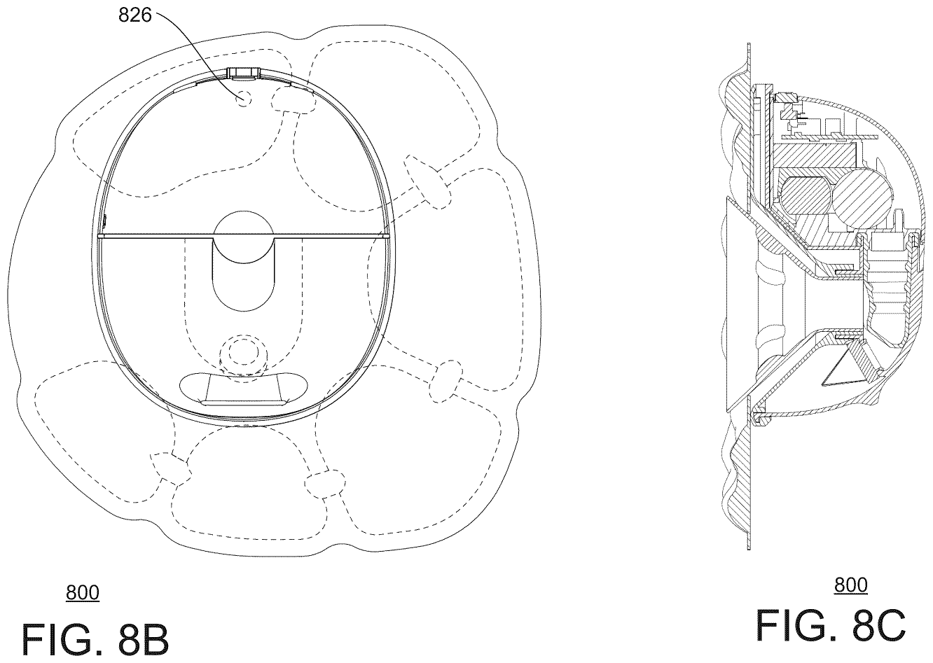

FIG. 8A illustrates an example massaging and suction system 800, in accordance with some embodiments. The massaging and suction system 800 includes various components, including a flange plug 810, massage cushions 820, a flange base 830, a controller device 840, a diaphragm 850, a collection container (such as a collection vessel 860), and a valve (such as a duckbill valve 870). In this example, the controller device 840 incorporates the control system 120, the second pump 118, and/or some or all of the sensors 114 as illustrated in FIG. 1. FIGS. 8B and 8C provide different perspectives of the assembled massaging and suction system 800 as illustrated in FIG. 8A. In the illustrated example, the flange plug 810 is used as a flange insert, which can be inserted into a flange for suction functions. Additionally, in this example, the flange base 830 serves as the flange.

In certain embodiments, the controller device 840 serves as the central processing and control hub of the massaging and suction system 800, managing and synchronizing both suction and massage functionalities. The controller device 840 is designed to be compact, lightweight, and ergonomically shaped to fit seamlessly within the pump assembly while ensuring case of use for the user. The controller device 840 houses electronic, mechanical, pneumatic, and user interface components, allowing for precise modulation of suction intensity, massage patterns, and real-time monitoring.

In certain embodiments, the controller device 840 includes various subsystems, such as a microcontroller unit (MCU) and control electronics, user interface, a suction assembly, a massage assembly, a power and battery system, and wireless connectivity and smart features. The MCU is configured to process user inputs, regulate suction and massage cycles, and execute preprogrammed and/or adaptive algorithms to optimize pumping efficiency. For example, the MCU controls suction power and rhythm by managing the vacuum pump and solenoid valve sequences. The MCU regulates massage intensity and cycle timing, to ensure smooth inflation and deflation of the massage cushions 820. Additionally, the MCU receives and processes sensor data (e.g., monitoring milk flow, suction pressure, temperature, and positioning) to adaptively adjust settings for improved efficiency and user comfort. The MCU also manages power distribution to various components, optimizing battery life and reducing unnecessary power consumption. The control electronics are configured to provide circuit protection functions, including stabilizing power delivery, filtering out electrical noise, preventing fluctuations, and protecting internal components from overload or overheating.

In certain embodiments, the controller device 840 implements an intuitive UI that allows the user to manually adjust suction and massage settings. The UI may include various hardware and/or software components, enabling the user to interact with the massaging and suction system 800 through various methods. For example, the UI may include one or more physical buttons, such as push-buttons or capacitive touch buttons allow control of suction intensity, massage cycle settings, and power on/off functionality. In some examples, the UI may include a touchscreen display (e.g., a liquid-crystal display (LCD) or organic light-emitting diode (OLED) touchscreen) that provides a visual interface for users to view real-time data, set preferences, and customize operational parameters. Additionally and/or alternatively, the UI may integrate a mobile application that runs on a terminal device, such as a smartphone, a personal computer, a smart watch, etc. For example, the controller device 840 may wirelessly connect to a smartphone application, enabling remote adjustments, tracking of pumping sessions, and access to personalized insights based on historical pumping data. The display or the application interface may show various information, including current suction level and mode (continuous or pulsating), massage mode, cycle intensity, and duration, battery percentage and charging status, wireless connectivity status, and/or real-time sensor feedback, such as milk flow rate and pressure levels. In certain embodiments, the display or the application interface may visualize some all of the content in the UI 700 as illustrated in FIG. 7.

The suction assembly includes one or more vacuum pumps (e.g., the second pump(s) 118 as shown in FIG. 1) and one or more solenoid valves. The suction assembly is configured to generate and regulate vacuum pressure necessary for extracting milk. In certain embodiments, a compact, high-efficiency diaphragm pump is used to create negative pressure within the pump system, simulating the natural sucking action of an infant. The one or more vacuum pumps can be configured with adjustable suction levels, enabling catering to different comfort preferences. The one or more vacuum pumps can provide consistent vacuum pressure under the selected mode, preventing fluctuations that could hinder milk extraction. Additionally, the one or more vacuum pumps may be designed to operate with low noise, allowing for discreet use in public or workplace settings. In certain embodiments, the one or more solenoid valves are electronically controlled to open and close at pre-determined intervals, regulating the pulsation of suction cycles to mimic a baby's suck-and-swallow pattern. The one or more solenoid valves may be configured to incorporate fine-tuned frequency control that enables adjustment of suction rhythms in real-time, gradual release of suction that prevents discomfort or nipple trauma, and pressure stabilization that ensures uniform suction application. The vacuum pump and solenoid valve system are pneumatically connected to the diaphragm and flange assembly (e.g., through the diaphragm 850, the flange base 830, and the flange plug 810), which transmits vacuum pressure directly to the breast for efficient milk extraction.

The massage assembly is a pneumatic system independent from the suction assembly, which is configured to inflate and deflate the massage cushions 820. The massage assembly includes one or more air pumps (e.g., the first pump(s) 124 as shown in FIG. 1) and one or more solenoid valves. In certain embodiments, the one or more air pumps include a miniature air pump. The one or more air pumps inflate the massage cushions 820 through precisely regulated airflow. The one or more air pumps may be configured with low power consumption to prolong battery life. The pump(s) may operate with rapid inflation and deflation cycles to ensure effective stimulation. Additionally, the pump(s) may adopt a compact design, allowing the pump(s) to fit within the limited space of a wearable device (e.g., the massaging and suction system 800). The one or more solenoid valves are used for airflow regulation. The valve(s) control the direction and intensity of air movement into and out of the massage cushions 820. In certain embodiments, the one or more solenoid valves are configured to provide sequential inflation and deflation, creating a wave-like compression motion. The valve(s) may enable variable compression intensities, allowing users to select massage pressure levels. Additionally, the valve(s) may provide independent channel control, enabling customized patterns that target different areas of the breast.

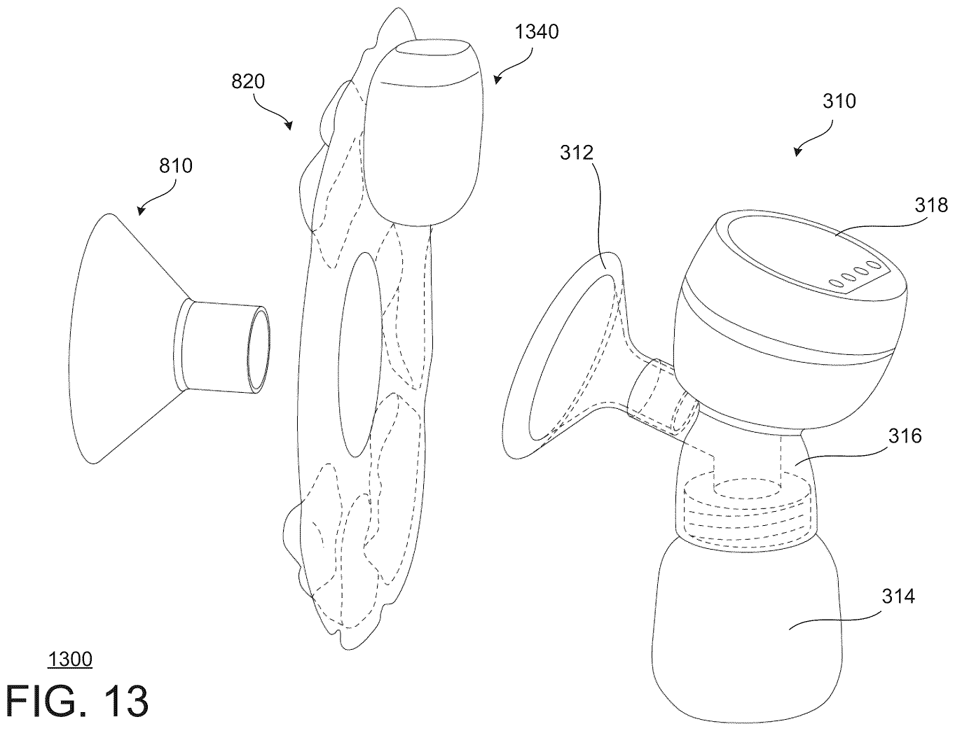

In certain embodiments, as an alternative, the controller device is detachable and plugs directly into the massage cushions 820, such as the controller device 1340 illustrated in FIG. 13. This design allows the massage cushions 820 to be used in conjunction with any standard or wearable breast pump, such as the breast pump system 310 described with reference to FIG. 3A. As shown in FIG. 13, the flange plug 810, e.g., used as a flange insert, can be inserted into the flange 312 of the breast pump system 310. In this configuration, the massage cushions 820 is positioned and secured between the flange plug 810 and the flange 312 after assembly. This setup provides flexibility for users opting for the standalone massage cushion system. In certain embodiments, the controller device 1340 includes only the massage assembly and related components. Alternatively, the controller device 1340 includes both the massage assembly and the suction assembly, along with other suitable components.

The controller device described herein, such as the controller device 840 or 1340, presents several advantages over conventional breast pump control units. For example, the controller device according to certain embodiments adopt a compact and wearable design. Traditional controllers typically require external tubing and bulky housings. In contrast, the controller device is integrated within the pump system, ensuring seamless hands-free operation. In some examples, the controller device according to certain embodiments enables synchronized suction and massage, which is enabled by precisely coordinating vacuum cycles with massage inflation. This approach maximizes efficiency and comfort. In some instances, the controller device according to certain embodiments allows for user-customizable settings. For example, users can fine-tune the system to match their comfort level through the physical interface and/or mobile application. In some variations, the controller device according to certain embodiments adopts a cross-compatibility design featuring an independently operable massage assembly. The standalone massage configuration allows the massage cushions to be used with any standard or wearable pump, expanding the massaging and suction system's usability beyond a single device. Furthermore, the controller device according to certain embodiments may incorporate noise-reducing components ensure that the device operates discreetly, making it suitable for public use. Additionally, the controller device according to certain embodiments may integrated sensors and artificial intelligence (AI) driven insights, enabling the pump to dynamically adjust to optimize milk expression while maintaining user comfort.

Referring back to FIG. 8A, the massaging and suction assembly 110 can be assembled from a plurality of discrete components, including the flange plug 810, the massage cushions 820, and optionally the flange base 830. The flange plug 810 is a conical insert that stabilizes the massage cushions 820 and adapts for different nipple sizes. The flange plug 810 is used as a flange insert according to at least one embodiment. In the illustrated example, the flange base 830 provides a contoured interface that connects to the breast and directs milk flow into the collection vessel 860. The massage cushions 820 include inflatable chambers (or bladders) designed to mimic hand compression, enhancing milk ejection through rhythmic stimulation (or other suitable stimulations) using the controller device 840.

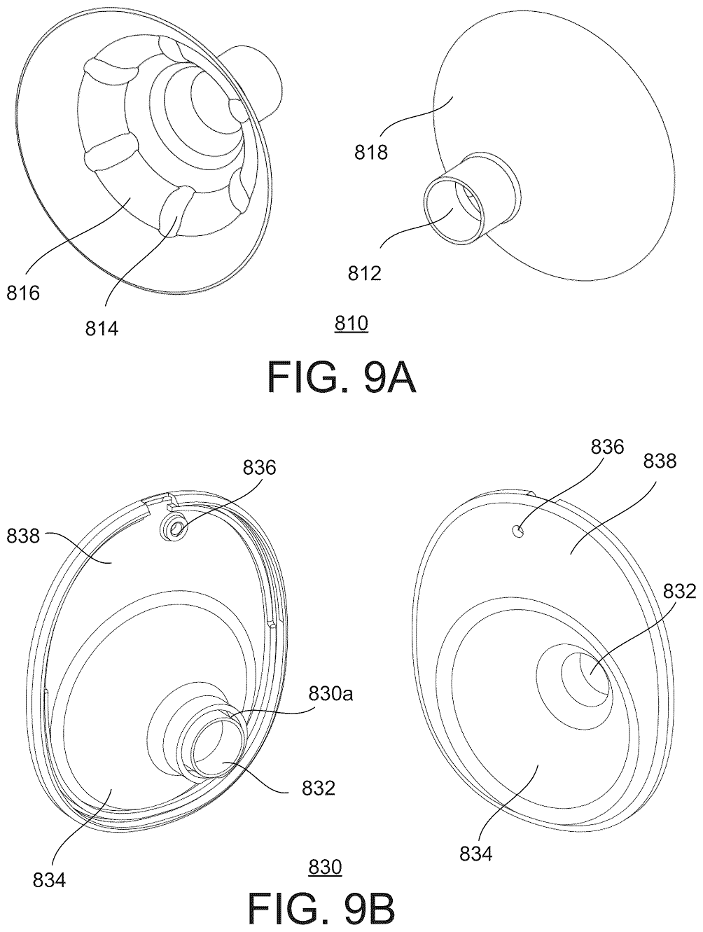

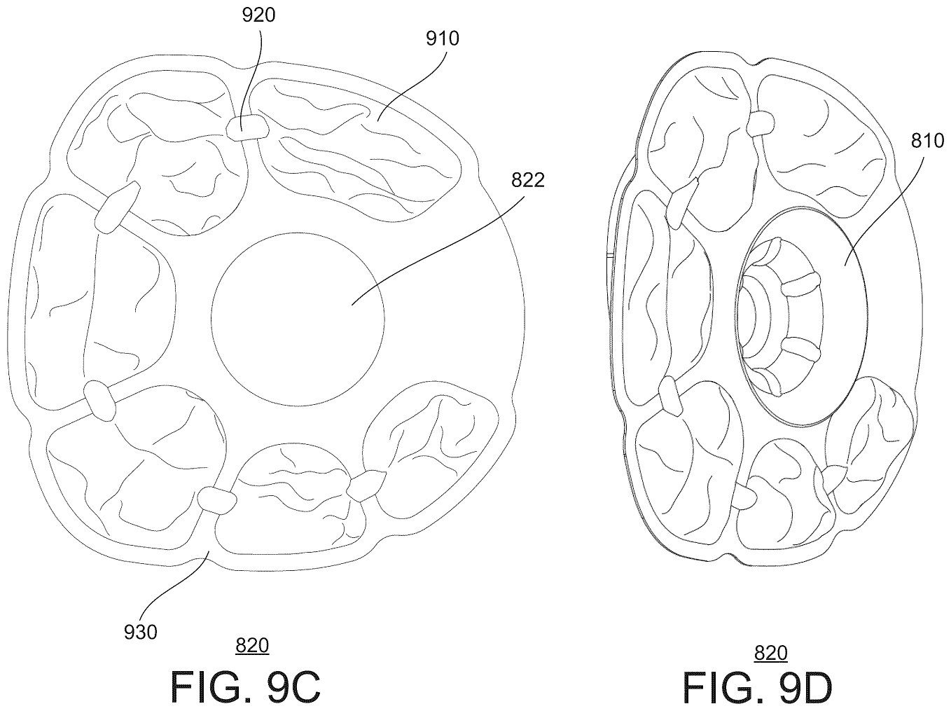

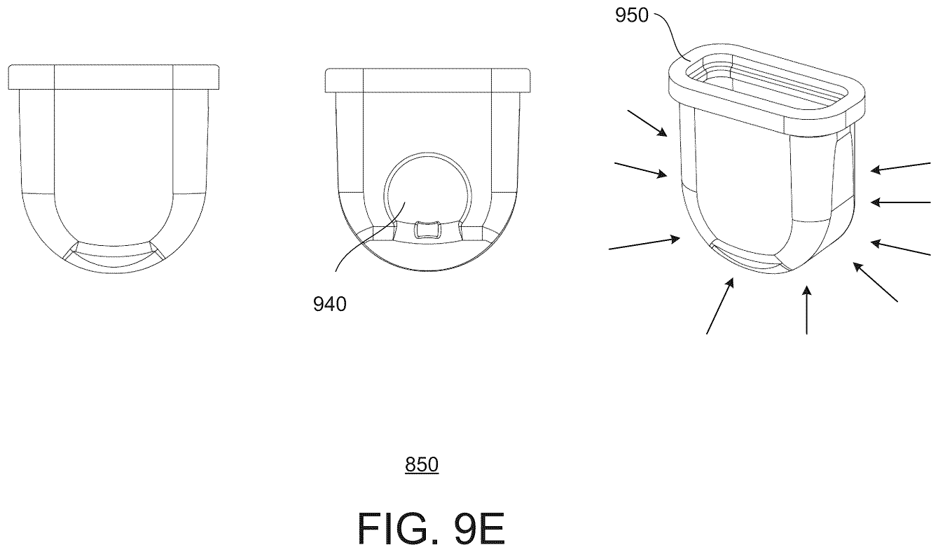

FIGS. 9A and 9B provide different perspectives of the flange plug 810 and the flange base 830, respectively, in accordance with some embodiments.

As shown in FIG. 9A, the flange plug 810 is a conical-shaped component. The flange plug 810 may be designed with a conical-shaped body 818 that tapers towards an opening 812. The flange plug 810 is designed to support and stabilize the massage cushions 820, ensuring that the massage cushions 820 function effectively during operation. Unlike conventional flange plugs/inserts that merely serve as an interface between the flange and the breast, the flange plug 810 actively contributes to the structural integrity of the massage system (e.g., the massaging cushions 820), ensuring proper alignment and enhancing user comfort.

The flange plug 810 may fit snugly within the flange base 830 or another suitable flange, forming a supportive layer between the breast and the pump mechanism. The flange plug 810 serves multiple functions, including (i) holding the massage cushions 820 securely in place, preventing misalignment or displacement during operation, (ii) providing a structured interface for air distribution, ensuring that the inflation and deflation cycles of the massage cushions 820 occur as intended, and (iii) facilitating proper suction transmission, allowing vacuum pressure from the diaphragm system (e.g., including the diaphragm 850) to remain stable and efficient.

In certain embodiments, the flange plug 810 is a conical insert that integrates a ring of small, flexible nodules 814 positioned within the inner circumference of the insert, designed to provide additional tactile stimulation. The nodules 814 may enhance massage effectiveness by applying localized pressure around the areola, mimicking manual compression techniques known to stimulate milk ejection. Furthermore, the nodules 814 may contribute to the stability of the flange plug 810, ensuring that the flange plug 810 retains its shape even under the dynamic pressure changes caused by the inflation and deflation of the massage cushions 820. Additionally, the nodules 814 may provide a secondary layer of comfort, allowing for a gentle yet firm interface between the breast and the pump mechanism. The structure of the flange plug 810 and/or the nodules 814 may be adapted for (i) breast tissue massage and stimulation tools, aiding in circulation enhancement and lymphatic drainage, (ii) postpartum breast therapy, assisting in recovery and reducing engorgement discomfort, and (iii) non-lactation compression therapy, providing gentle but firm support in medical-grade vacuum treatment devices.

In certain embodiments, the inner layer of the flange plug 810 may incorporate a structured ring 816, which serves as an additional support element for the massage cushions 820. This ring 816 may reinforce the alignment of the massage cushions 820, ensuring uniform compression across the breast. Moreover, the ring 816 may distribute inflation pressure more evenly, preventing isolated pressure points that could lead to discomfort. Furthermore, the ring 816 may help maintain vacuum integrity, ensuring that air pathways remain unobstructed during operation.

In certain embodiments, the flange plug 810 is constructed from medical-grade silicone or a similar biocompatible elastomer. These materials feature a soft and flexible composition, providing comfort while securing the massage cushions 820 in place. Additionally, these materials have hypoallergenic and non-toxic properties, minimizing skin irritation for extended wear. Furthermore, these materials provide durability under continuous suction and pressure cycles, preventing deformation and material fatigue. These materials are also sterilization compatibility, allowing for easy and effective cleaning through boiling, steam, or chemical sanitization.

In certain embodiments, the flange plug 810 may include reinforced edges or dual-layered construction to enhance stability and longevity, particularly in high-stress areas where the insert (e.g., the flange plug 810) interfaces with the massage cushions 820.

The flange plug 810 enhances both suction and massage efficiency, ensuring that users experience optimal milk extraction without unnecessary discomfort. For example, the flange plug 810 can achieve reduced air leakage that ensures consistent suction application, minimized misalignment issues that allows massage cushions 820 to function as intended, and enhanced nipple comfort facilitated by friction-minimizing surfaces of the flange plug 810, which prevent excessive chafing or pressure buildup.

To accommodate a diverse range of users, the flange plug 810 may be designed in multiple variations. In at least one embodiment, the flange plug 810 may be provided in various sizes. For example, adjustable diameter inserts may be implemented, allowing users to customize the internal nipple opening to fit their needs and prevent discomfort from improper sizing while ensuring effective suction. Ergonomic angled inserts may also be introduced, providing a natural forward tilt that improves alignment with the breast and reduces strain on the user's posture during pumping sessions. Soft-rimmed inserts with enhanced cushioning may further be included, featuring a cushioned outer rim for additional comfort, particularly ideal for users with sensitive breast tissue or those prone to nipple soreness. In at least one embodiment, the flange plug 810 may be fitted with one or more flange inserts. For example, a universal flange plug may be provided and used in conjunction with one or more additional flange inserts, such as the variant inserts described herein, to accommodate a diverse range of users.

As shown in FIG. 9B, the flange base 830, includes a conical-shaped portion and a rim extended from one end of the conical-shaped portion. The flange base 830 serves as the primary interface between the user and the pumping system, ensuring a secure and comfortable seal against the breast. The flange base 830 is utilized to ensure effective suction transmission, efficient milk flow, and overall user comfort during pumping sessions.

The flange base 830 is designed with a conical-shaped body 834 that tapers towards an opening 832, which aligns with the nipple to facilitate milk expression. The structure of the flange base 830 includes the conical-shaped body 834, a contoured rim 838, the opening 832, a flexible shaft or connector 830a at the rear, and an opening 836 located in the upper section (e.g., of the rim 838) above the conical-shaped body 834. The contoured rim 838 adheres to the massage cushion 820, providing ergonomic support and ensuring an airtight seal. In certain embodiments, the opening 832 is a centrally positioned opening designed to accommodate different nipple diameters, ensuring comfortable positioning. However, it should be noted that the opening 832 can be positioned elsewhere in the flange base 830. The flexible shaft or connector 830a at the rear allows attachment to the diaphragm housing (e.g., in the collection vessel 860) and/or the collection vessel 860.

The opening 836 is configured to facilitate the connection of the massage cushions 820 to the control system (e.g., the controller device 840), enabling air or fluid pathways necessary for the massage functionality. The placement of the opening 836 ensures that the massaging system (e.g., the massage cushions 820) operates efficiently without interfering with the suction mechanism (e.g., including the collection vessel 860 and the components incorporated therein) or compromising the seal between the flange base 830 and the collection vessel 860. This feature enhances the overall integration of the massage and suction system 800 and contributes to the pump's multifunctional efficiency. However, it should be noted that the opening 836 can be positioned elsewhere in the flange base 830, and more than one opening 836 can be provided. Furthermore, in some embodiments, the flange base 830 may not include any openings. Instead, the massage cushions 820 may be connected to the air pump in the controller device 840 via external connections, such as tubing.

The flange base 830 may be constructed from medical-grade silicone or similar biocompatible elastomers, which have soft, flexible material properties, enabling conformation to different breast shapes and sizes. Additionally, these materials provide hypoallergenic and non-toxic surfaces, preventing irritation and ensuring safety for repeated skin contact. Furthermore, the flange base 830 made of these materials exhibit high durability, resistant to deformation under continuous suction pressure and prolonged use. These materials also have sterilization compatibility, allowing for safe cleaning using boiling, steam, or chemical sanitization. In certain embodiments, the flange base 830 may also include a rigid structural reinforcement layer, ensuring stability and optimal suction distribution while maintaining user comfort.

The flange base 830 ensures uniform suction distribution, minimizing discomfort while maximizing milk extraction. Additionally, the flange base 830 serves as a secure and leak-proof attachment, preventing loss of vacuum pressure during operation. In certain embodiments, the flange base 830 may adopt an adaptive fit for various breast shapes, reducing localized pressure points that may cause discomfort.

In certain embodiments, the flange base 830 is engineered to “snap” securely into the back of the collection vessel 860, creating an airtight, leak-proof seal that is crucial for maintaining optimal vacuum levels during pumping. This snap-fit mechanism utilizes the inherent flexibility of silicone or similar materials, allowing for easy attachment and removal without compromising the integrity of the seal. The flange base 830 also supports the overall compactness of the pump, contributing to its discreet, low-profile form factor. Additionally, the seamless integration of the flange base 830 with the collection vessel 860 reduces the risk of milk leakage, improves hygiene, and simplifies cleaning. The durable construction of the flange base 830 resists deformation from repeated use, sterilization, and varying pressure cycles, ensuring reliable performance over time.

In the example as shown in FIG. 8A, the flange base 830 interfaces with multiple elements of the massaging and suction system 800, ensuring seamless operation. For example, the flange base 830 integrates with the diaphragm system (e.g., including the diaphragm 850), which regulates suction flow while maintaining vacuum stability. Additionally, the flange base 830 integrates with the collection vessel 860, ensuring efficient transfer of expressed milk without leakage. Furthermore, the flange base 830 integrates with the flange plug 810, which acts as a stabilizing insert for the massage cushions 820, providing added customization for different nipple sizes.

The flange base 830 can be designed in multiple variations to accommodate user preferences and anatomical differences. In one example, the flange base 830 may be designed as a soft-fit flange with an adjustable rim. This design features a flexible, cushioned rim to enhance comfort during extended use, and adapts to variations in breast tissue firmness, reducing the risk of discomfort or redness. In another example, the flange base 830 may accommodate interchangeable flange plugs, allowing users to swap different nipple diameter inserts, ensuring an optimized fit for individual needs. This design prevents incorrect sizing, which can lead to discomfort or inefficient milk extraction. In yet another example, the flange base 830 may be designed with a slight forward tilt (e.g., to be used with an angled flange for enhanced comfort), thereby improving alignment, and promoting natural milk flow. This design may reduce strain on the user's shoulders and back by enabling a more relaxed posture.

During operation, the flange base 830 can reduce suction turbulence, maintaining a steady and gentle pressure gradient; minimize noise and vibration, enhancing discreet operation; and prevent milk backflow, while working in conjunction with the diaphragm 850 and the duckbill valve 870 to maintain hygiene and efficiency.

In certain embodiments, the flange base 830 may be adapted for therapeutic breast massage that incorporates heated or vibrational elements to aid in milk flow stimulation and mastitis prevention. The flange base 830 may also be used in medical applications, such as post-surgical compression therapy for breast tissue recovery, and as lactation training tools to help new mothers establish proper pumping and breastfeeding techniques.

FIGS. 9C and 9D provide different perspectives of the massage cushions 820, in accordance with some embodiments.

The massage cushions 820 are configured to simulate natural hand compression techniques, enhancing milk ejection by applying gentle, rhythmic pressure around the breast. Unlike traditional suction-based breast pumps that rely solely on vacuum pressure to extract milk, the inclusion of massage cushions 820 improves comfort, stimulates letdown, and enhances overall pumping efficiency.

Similar to the massaging and suction assembly 110 as shown in FIG. 2, the massage cushions 820 are formed by two layers, including a cushion layer 824 designed with a plurality of malleable ridges (e.g., the chambers) and a base layer 828, as illustrated in FIG. 8A. Different from the massaging and suction assembly 110 as shown in FIG. 2, the base layer 828 does not include a conical-shaped portion. Instead, the base layer 828 can be assembled with the flange plug 810 and/or the flange base 830 to serve a similar function.

In certain embodiments, the cushion layer 824 is also referred to as an inner layer, while the base layer 828 is also referred to as an outer layer. The outer layer 828 may be designed with higher hardness or increased thickness, ensuring controlled inflation that directs the pressure toward the breast rather than outwardly dispersing it. In certain embodiments, the outer layer 828 is incorporated with one or more ports (e.g., the opening 826, or a nozzle similar to the outlet 216 as shown in FIG. 2) through which air is introduced into the cushions from the massage assembly of the controller device 840. FIG. 9C shows the inner layer 824 of the massage cushions 820. The inner layer 824 is designed with the plurality of chambers 910, such as thermoformed “finger-like” projections, mimicking natural hand massage by exerting localized pressure when inflated. In certain embodiments, the surface of the chambers 910 may be designed with raised patterns (e.g., wrinkles or grooves). The plurality of chambers 910 are interconnected through channels 920. The inner layer and the outer layer are bonded together along a sealed perimeter 930, ensuring that air distribution occurs in a controlled manner without leaks.

In certain embodiments, the overall shape of the massage cushions 820 forms a cone with an opening 822, allowing them to be inserted between the flange base 830 and the flange plug 810. This conical structure may ensure that the massage cushions 820 fit securely within the flange components (e.g., the flange plug 810 and/or the flange base 830) while providing compression around the breast. The size of the cone can be adjusted or varied, accommodating different breast shapes and sizes for a customizable fit. The placement of the massage cushions 820 allows for hands-free pumping, making them suitable for wearing inside a bra for discreet, continuous operation.

To accommodate user preferences and anatomical differences, the massage cushions 820 may be designed in several variations. For example, the massage cushions 820 may be multi-zone massage cushions, which include separate inflation zones that target different areas of the breast. This allows users to adjust massage focus based on personal comfort and milk flow responsiveness. In some examples, the massage cushions 820 may be adjustable pressure cushions, which incorporate variable-thickness chambers 910, enabling selective pressure adjustments for personalized comfort. In some instances, the massage cushions 820 may be vibrational massage cushions, which incorporate one or more low-frequency vibration elements to enhance circulation and aid in milk letdown.

In certain embodiments, the massage cushions 820 are constructed from dual-layer thermoplastic polyurethane (TPU) film or a similar flexible, biocompatible material.

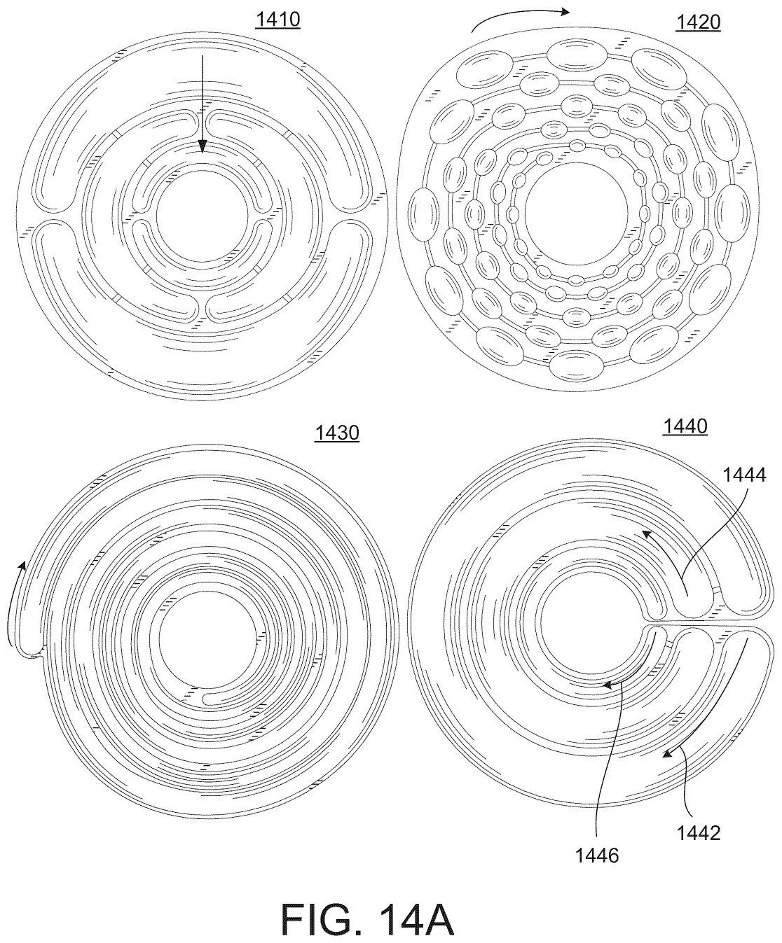

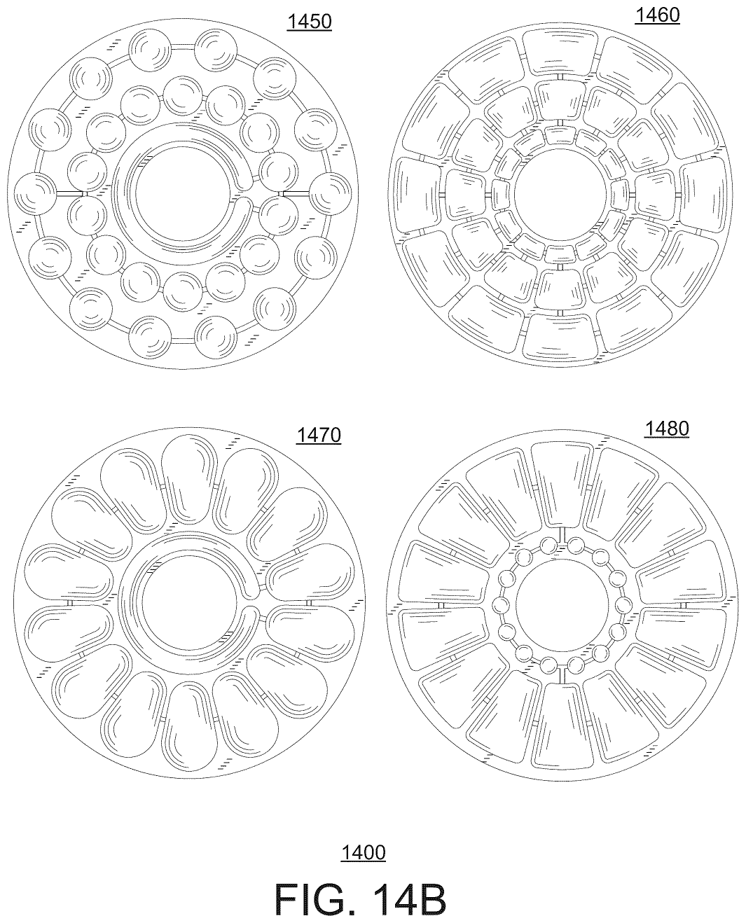

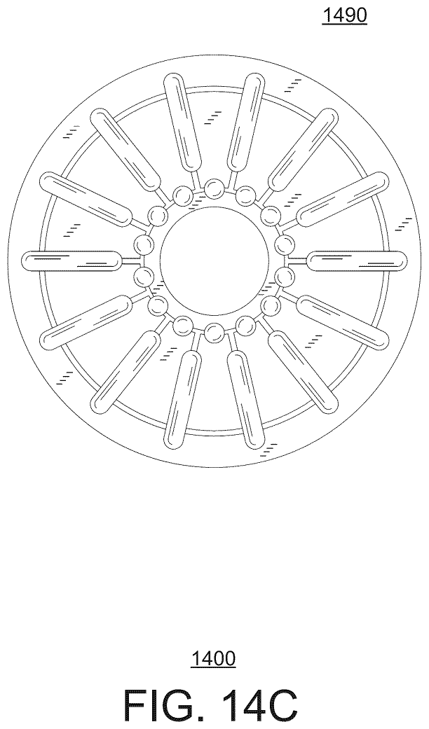

In certain embodiments, the inner and outer layers of the massage cushions 820 are heat-sealed together to form the chambers 910. In certain embodiments, the plurality of chambers 910 are sequentially inflated and deflated, creating a wave-like compression effect that encourages consistent milk flow. The chambers 910 may be of various shapes and sizes, including concentric pockets, spiral configurations, paired pockets, or a series of interconnected chambers, as illustrated in FIGS. 14A-14C. The inflation and deflation pattern of these various embodiments can create different sensations, allowing for customizable massage experiences.

Referring back to FIG. 8A, the massage cushions 820 are configured to fit between the flange base 830 and the flange plug 810, ensuring the massage cushions 820 remain securely positioned throughout pumping/suction. The massage cushions 820 are configured to provide a dynamic massaging action that complements the pumping/suction process, promoting increased milk expression by stimulating milk ducts through gentle pressure. Additionally, the massage cushions 820 provide enhanced comfort by reducing the reliance on high suction levels, which can cause discomfort over time. Furthermore, the massage cushions 820 provide optimized letdown response, mimicking the natural stimulation provided by a nursing infant.

In the illustrated example, the massage cushions 820 are integrated into the massaging and suction system 800 in the following sequence: the flange base 830→the massage cushions 820→the flange plug 810. This ensures that all components remain properly positioned throughout the pumping session. As shown in FIG. 8A, the opening 832 of the flange base 830, the opening 822 of the massage cushions 820, and the opening 812 of the flange plug 810 are aligned during assembly. FIG. 9D shows the result of the flange plug 810 being inserted into the opening 822 of the massage cushions 820. The flange plug 810 provides structural support, ensuring that the massage cushions 820 maintain their shape and position during operation. However, it should be noted that the flange plug 810, the massage cushions 820, and/or the flange base 830 can be replaced with components, or additional components can be used. In at least one embodiment, one or more additional flange inserts may be placed inside the flange plug 810. In another embodiment, the massage cushions 820 may be replaced with cushions of different designs, such as those shown in FIGS. 14A-14C, allowing users to select a configuration that best suits their needs. Additionally, in the example shown in FIG. 13, the flange base 830 may be omitted, with the flange plug 810 inserted directly into the flange 312 to achieve a similar connection effect.

In certain embodiments, the massage cushions 820 are connected to the controller device 840 via flexible tubing that passes through the flange base 830 (e.g., through the opening 836 in the flange base 830), allowing precisely regulated airflow to enter and exit the massage cushions 820. In certain embodiments, the massage cushions 820 are connected to the controller device 840 via flexible tubing that bypasses the flange base 830. For example, the flange base 830 may either lack an opening or have an opening that is not utilized. In certain embodiments, the flexible tubing may also be incorporated into the design of a “nozzle,” for example, placed at the opening 826 on the outer layer 828 of the massage cushions 820.

In certain embodiments, the airflow management associated with the massage cushions 820 may be facilitated through (i) one or more inflation nozzles (or the opening 826), which allow air to enter the chambers 910 in a controlled manner, (ii) deflation pathways, which enable rapid decompression to reset the massage cushions 820 for the next compression cycle, and (iii) the interconnected chamber design (e.g., through one or more channels 920), which enables progressive inflation to create an optimal wave-like massage effect.

In certain embodiments, the placement and number of nozzles/openings may be designed to regulate airflow direction, ensuring that each chamber 910 inflates in a predefined sequence. Additionally, precise control of inflation and deflation cycles may be realized, ensuring uniform compression around the breast. Furthermore, customized massage patterns may be facilitated, enabling users to experience varied sensations depending on the sequence and intensity of compression. Adaptive responsiveness may also be achieved, adjusting the massage pattern based on user-selected settings in a control interface (e.g., the UI 700 as shown in FIG. 7).

In certain embodiments, the massage cushions 820 are designed to fit comfortably within a bra, making them ideal for hands-free pumping while ensuring gentle, all-around breast massage that promotes effective milk expression.

The massage cushions 820 may reduce suction dependency, ensuring efficient milk expression with minimal vacuum pressure. Additionally, the massage cushions 820 may prevent over-compression, for example, by using calibrated airflow control to maintain consistent comfort levels. Furthermore, the massage cushions 820 may provide easy cleaning and maintenance, as the massage cushions 820 may utilize smooth, non-porous materials that resist bacterial buildup.

The massage cushions 820 may be used for lactation support. Additionally, the massage cushions 820 can be adapted for (i) postpartum breast therapy, aiding in the relief of engorgement and clogged ducts, (ii) lymphatic drainage applications, improving circulation and reducing breast discomfort, and (iii) general breast massage therapy, promoting tissue elasticity and long-term breast health.

FIG. 9E provides different perspectives of the diaphragm 850, in accordance with some embodiments. The diaphragm 850 is a vacuum-sealing component within the massaging and suction system 800, designed to facilitate the creation and maintenance of negative pressure essential for efficient milk extraction. The diaphragm 850 acts as a flexible barrier, allowing the controlled transmission of suction force from the vacuum pump to the flange assembly (e.g., including the flange plug 810 and/or the flange base 830) while preventing milk backflow and contamination. As shown in FIG. 8A, the diaphragm 850 is connected to the suction assembly on one side (e.g., the pumps in the controller device 840 for generating movement) and to the flange assembly (e.g., including the flange base 830 and/or the flange plug 810) on the other (to control airflow and vacuum pressure).

In certain embodiments, the diaphragm 850 includes a thin-walled membrane 940 with a variable thickness gradient, providing optimized flexibility in certain areas while maintaining structural integrity. In certain embodiments, the edges of the diaphragm 850 are reinforced with a thicker rim 950, ensuring a secure fit within the collection vessel 860 while resisting deformation over time.

During operation, the diaphragm 850 moves/flexes up and down (or flexes back and forth or flexes in and out), thereby creating changes in air pressure that result in the rhythmic suction needed for effective milk expression. In the “U” shape embodiment as shown FIG. 9E, the diaphragm 850 compresses/contracts inwards, which allows a larger displacement of vacuum/air to compress, thereby increasing the intensity and/or amount of suction that the diaphragm 850 is able to produce (arrows show the compression direction). The diaphragm 850 forms an airtight seal within the pump to maintain consistent vacuum levels without leaks. The diaphragm 850 prevents breast milk from coming into contact with the pump's internal mechanical parts, reducing the risk of mold or bacteria buildup. Furthermore, the diaphragm 850 prevents milk from flowing back up the tubing and into the suction assembly and the controller device 840.

As illustrated in FIG. 8A, the diaphragm 850 is securely housed within a dedicated compartment 864 inside the collection vessel 860. The sealing interface is designed to (i) maintain an airtight seal, preventing air leaks that could disrupt suction performance, (ii) separate the vacuum mechanism from the milk reservoir, ensuring that no milk enters the pneumatic system, and (iii) facilitate quick assembly and removal, allowing for easy cleaning and maintenance.

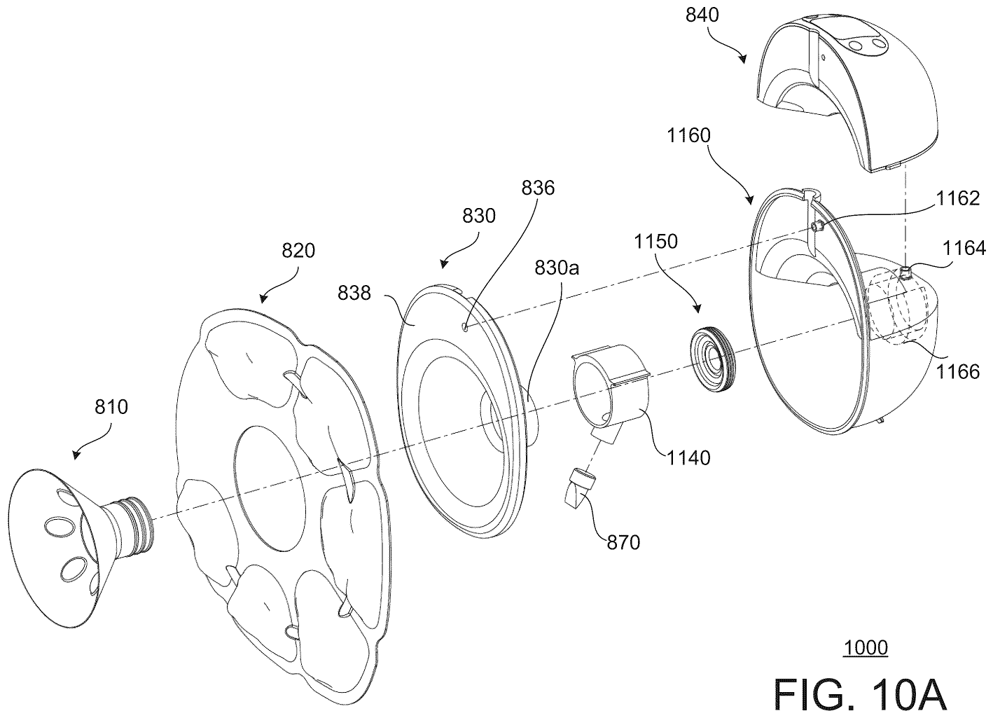

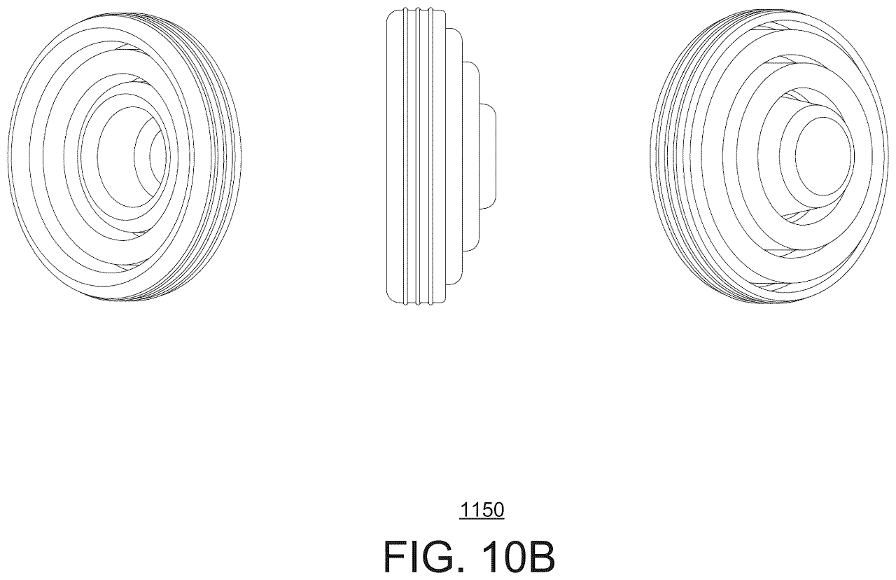

The diaphragm can be designed in multiple configurations to optimize performance based on different user preferences and anatomical considerations. In some examples, the diaphragm may be designed as a circular dome diaphragm, which has a uniformly curved profile with central expansion. This design distributes suction force evenly across the flange, ensuring consistent vacuum levels. The circular dome diaphragm may be used for general pumping applications, providing a balance of comfort and efficiency. In some instances, the diaphragm may be designed as a tongue-shaped (U-shaped) diaphragm (e.g., the diaphragm 850 as shown in FIG. 9E), which has an elongated, asymmetric structure, allowing for greater displacement of vacuum volume. This design enhances dynamic air movement, producing a more pronounced rhythmic suction. The U-shaped diaphragm may be beneficial for users requiring stronger vacuum action or for those with low milk supply. In some variations, the diaphragm 850 may be designed as a multi-layered diaphragm with reinforced zones (e.g., the diaphragm 1150 as shown in FIG. 10B), which incorporates variable material thickness to create differential flexion points. This design enables targeted vacuum force distribution, optimizing milk flow while reducing nipple discomfort. The multi-layered diaphragm may provide extended durability, especially for users requiring frequent pumping sessions.

The diaphragm, such as the diaphragm 850, operates through cyclic flexion, responding dynamically to pressure variations generated by the vacuum pump (e.g., in the controller device 840). In certain embodiments, during suction, the diaphragm deforms, such as by expanding outward, flexing back and forth, or flexing in and out, to create a negative pressure zone that draws milk through the flange (e.g., the flange plug 810) and into the collection vessel 860. When suction is released, the diaphragm retracts back to its neutral position, restoring equilibrium and preparing for the next cycle. This controlled motion ensures continuous and consistent suction cycles, mimicking the natural feeding rhythms of an infant.

In certain embodiments, the diaphragm is configured to operate within a precisely controlled vacuum pressure range. This ensures (i) gentle but effective suction, reducing the risk of nipple trauma, (ii) stable vacuum retention, minimizing fluctuations that could disrupt milk flow, and (iii) adaptive response to suction adjustments, enabling users to fine-tune comfort levels without compromising efficiency.

In certain embodiments, the diaphragm 850 is configured to work seamlessly with a sensor array (e.g., one or more sensors 114 as depicted in FIG. 1), allowing for real-time monitoring of suction force, diaphragm movement patterns, and overall vacuum performance. If deviations are detected, the controller device 840 can automatically adjust pump intensity, optimizing user experience and efficiency.

In certain embodiments, the diaphragm 850 may be constructed from medical-grade silicone or a comparable biocompatible elastomer. As such, the diaphragm 810 may be highly flexible and elastic, allowing for repeated expansion and contraction without material degradation. Additionally, the diaphragm 810 may be resistant to tearing, warping, and compression fatigue, ensuring prolonged durability under continuous operation. Furthermore, the diaphragm 810 may be non-porous and hypoallergenic, minimizing bacterial retention and ensuring hygienic operation. The diaphragm 810 may also be compatible with standard sterilization methods, including boiling, steam sterilization, and chemical disinfection.

The diaphragm 850 may be used in breast pumping applications, as well as in other suitable systems/applications. For example, the diaphragm 850 may be adapted for medical vacuum systems, where controlled suction is required for wound care or respiratory therapy, wearable therapeutic devices, such as lymphatic drainage systems that require rhythmic compression cycles, and breast health monitoring systems/applications, which integrate pressure-sensitive elements to detect abnormalities in breast tissue elasticity over time.

Referring back to FIG. 8A, the duckbill valve 870 is a one-way check valve designed to regulate the flow of expressed milk within the massaging and suction system 800. The duckbill valve 870 allows milk to pass into the collection vessel 860 while preventing backflow, thereby ensuring a hygienic and efficient pumping process. In this example, the duckbill valve 870 is seamlessly integrated into the collection vessel 860, maintaining optimal suction conditions throughout the pumping cycle. The duckbill valve 870 includes a flattened, tapered body that terminates in a self-scaling slit. The slit remains closed under neutral pressure but opens when suction is applied, enabling milk to pass through. Once the suction is released, the elasticity of the material ensures that the slit immediately seals shut, preventing any milk from re-entering the pump's suction pathway. The self-scaling mechanism ensures that the pump maintains an airtight system, which is essential for consistent suction levels and user comfort. Unlike conventional membrane-based check valves, the duckbill design eliminates the need for multiple moving parts, reducing failure points and enhancing reliability.

In certain embodiments, the duckbill valve 870 is positioned at the base of the diaphragm 850, forming a sealed junction between the milk flow path and the collection vessel 860. The integration of the duckbill valve 870 may follow a press-fit or snap-in connection, enabling (i) a secure, leak-proof seal between components, (ii) quick and easy removal for cleaning and replacement, and (iii) compatibility with varying milk flow rates, accommodating both slow and fast let-down responses.

In certain embodiments, the duckbill valve 870 may function optimally under a range of suction pressures, allowing the massaging and suction system 800 to adapt to different pumping intensities without compromising efficiency. The duckbill mechanism ensures that even under low-suction settings, the valve responds dynamically to maintain smooth milk flow.

In certain embodiments, the duckbill valve 870 is made from medical-grade silicone or a similar biocompatible elastomer. As such, the duckbill valve 870 can be flexible and durable, allowing repeated compression and release without material fatigue. Additionally, the duckbill valve 870 may be resistant to deformation, ensuring consistent one-way flow over prolonged use. Furthermore, the duckbill valve 870 may be easy to clean and sterilize, compatible with boiling, steam, and chemical sterilization.

In certain embodiments, the valve (e.g., the duckbill valve 870) may implement other designs, such as multi-slit designs, which may allow for higher milk flow capacity while maintaining backflow prevention, reinforced edges, which may enhance the valve's ability to withstand extended use without warping, and pressure-sensitive variants, which adjust opening resistance based on the user's selected suction level.

In certain embodiments, the valve (e.g., the duckbill valve 870) may be designed with a textured or ribbed outer surface to improve grip during installation and removal, further enhancing user convenience.

In certain embodiments, the collection vessel 860 incorporates a compartmentalized design where the diaphragm 850 and duckbill valve 870 are positioned within designated airtight sections. This design ensures efficient vacuum retention, optimizing suction performance, backflow prevention, keeping expressed milk separate from the pump's internal components, and hygienic separation, preventing contamination by ensuring that milk does not come into contact with the pump's motorized elements.

The collection vessel 860 serves as a sealed containment unit for the expressed milk while integrating with the diaphragm and duckbill valve 870 to maintain vacuum stability. The collection vessel 860 is designed to be compact, lightweight, and easily detachable, the collection vessel 860 allows for efficient milk storage and hygienic handling during and after pumping. The collection vessel 860 is designed to securely connect with the pump assembly via a snap-fit or twist-lock mechanism. The collection vessel 860 is designed to ensure leak-proof operation during pumping, easy detachment for cleaning or milk transfer, and seamless alignment with the diaphragm 850 and duckbill valve 870 to prevent vacuum loss.

In certain embodiments, the collection vessel 860 is constructed from food-grade, BPA-free plastic, glass, or other medically safe materials that ensure safety and durability. As such, the collection vessel 860 can be transparent or semi-transparent to allow users to monitor milk volume in real time, impact-resistant to withstand accidental drops or physical stresses during use, temperature-resistant, making the collection vessel 860 suitable for refrigeration or warming without material degradation, and sterilization-compatible, allowing for cleaning through boiling, steam sterilization, or chemical sanitization without affecting its structural integrity.

In certain embodiments, the collection vessel 860 is calibrated with graduated volume markings (e.g., milliliters and ounces) to provide accurate milk volume readings. The markings may be embossed, printed, or laser-etched to ensure durability even after repeated cleaning and sterilization.

In certain embodiments, the collection vessel 860 is shaped to fit discreetly within a bra or pumping garment. For example, the collection vessel 860 may be designed with a slim, contoured profile that follows the natural curvature of the body, reducing bulkiness. Additionally, the collection vessel 860 may be designed with an optimized weight distribution to prevent discomfort during extended wear. Furthermore, the collection vessel 860 may be designed with a spill-proof, air-tight lid or cap for convenient milk storage post-expression.

In certain embodiments, the collection vessel 860 may incorporate one or more integrated cooling elements, such as a gel-based insulative layer, to keep milk fresh for an extended period before refrigeration. In certain embodiments, the collection vessel 860 may be collapsible or flexible, allowing users to minimize storage space when not in use. In certain embodiments, the collection vessel 860 may incorporate a built-in spout or dispensing system, enabling direct milk transfer into bottles or storage bags without requiring additional pouring steps.

The collection vessel 860 according to various embodiments of the present disclosure is engineered for efficiency, hygiene, and user convenience, ensuring a seamless and reliable milk expression process. The design enhances usability by allowing quick assembly and disassembly, making cleaning and reassembly effortless for busy lactating individuals.

In certain embodiments, the massaging and suction system 800 is integrated with one or more sensors to measure the amount of milk expressed, and/or rate of milk flow from the breast while pumping. It should be noted that any of the sensors discussed in the present application, along with other suitable sensors, can be integrated into the massaging and suction system 800. Additionally, the one or more sensors can be integrated at various locations in the massaging and suction system 800, such as in the massage cushions 820, the controller device 840, and more. In certain embodiments, data from the sensors can also be used as a feedback loop to the massaging and suction system 800, for example, to automatically adjust/modify the suction and massage settings depending on the milk flow.

In certain embodiments, beyond breastfeeding applications, the massage cushion system, such as the massage cushions 820 and a corresponding controller device (e.g., the controller device 840, 1340, etc.), can be adapted for other applications, including (i) post-surgical breast therapy, aiding in lymphatic drainage and reducing post-operative swelling, (ii) menopausal breast health, promoting circulation and comfort, and (iii) general pain relief for individuals with fibrocystic breast conditions.

FIG. 10A illustrates an example massaging and suction system 1000, in accordance with some embodiments. The massaging and suction system 1000 may share common components with the massaging and suction system 800 as illustrated in FIG. 8A. For example, the massaging and suction system 1000 may include the flange plug 810, the massage cushions 820, the flange base 830, the controller device 840, and a valve (such as the duckbill valve 870). Different from the massaging and suction system 800 as illustrated in FIG. 8A, in this example, the diaphragm 1150 is a multi-layered diaphragm with reinforced zones. The diaphragm 1150 is secured in a multi-way connector 1140. A first end of the multi-way connector 1140 is connected to the shaft or connector 830a of the flange base 830, a second end of the multi-way connector 1140 is connected to (or mounted onto) a connector base 1166 integrated inside the collection vessel 1160, and the end of the multi-way connector 1140 is connected to the duckbill valve 870. In this example, the suction assembly in the controller device 840 can provide suction force through the port 1164, while the massage assembly in the controller device 840 can provide airflow through the port 1162. The collection vessel 1160 includes a contoured rim matching the contoured rim 838 of the flange base 830, allowing the flange base 830 and the collection vessel 1160 to form an airtight seal when assembled. FIG. 10B provides different perspectives of the diaphragm 1150.

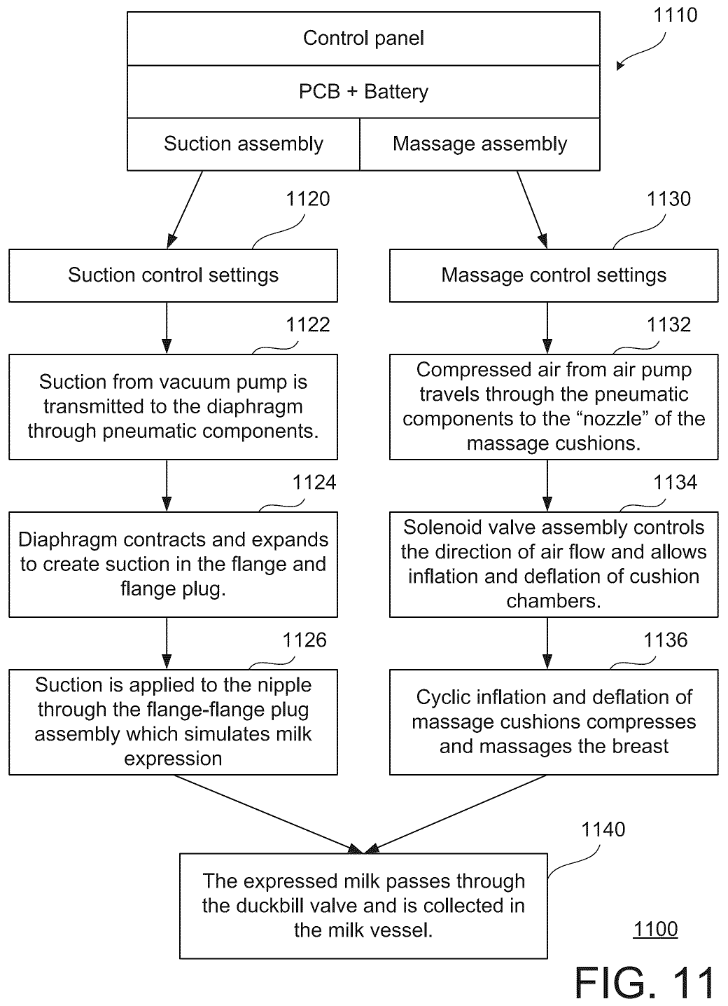

FIG. 11 is a flow diagram illustrating an example process 1100 performed by a massaging and suction system, in accordance with some embodiments. In certain embodiments, the massaging and suction system may include a controller device (e.g., the controller device 840) that incorporates both the massage assembly and the suction assembly. In certain embodiments, the massaging and suction system may include a controller device (e.g., the controller device 1340) that incorporates only the massage assembly. It should be noted the process may additionally or alternatively be performed by any one system, or any combination of systems, including, but not limited to, those described herein. Furthermore, some of the blocks described herein with respect to the process 1100 may be optional, and the blocks may be performed in different orders.

As shown in FIG. 11, a control system 1110 includes a control panel, a printed circuit board (PCB) integrated with control circuits, chips, and/or modules to facilitate various control functions, and a battery device/module to power the control system 1110. In this example, the control system 1110 is integrated with an air (or fluid) pump with a corresponding solenoid valve associated with massage functions (referred to as the suction assembly), and a vacuum (or suction) pump with a corresponding solenoid valve associated with suction functions (referred to as the massage assembly).

In certain embodiments, the control system 1110 is powered by a rechargeable lithium-ion or lithium-polymer battery, designed for extended operational use. In certain embodiments, the control system 1110 incorporates Bluetooth and/or Wi-Fi connectivity, enabling seamless integration with a mobile application. Smart features may include adaptive suction & massage control, data logging and analytics, personalized recommendations, and remote operation. As such, the massaging and suction system (or the control system 1110 integrated therein) may dynamically adjust suction and massage patterns based on real-time sensor feedback, ensuring optimal efficiency for each session. Users can track milk production, session duration, and historical trends via the application. AI-powered algorithms provide suggestions for optimizing suction levels and massage intensity based on past usage data. Furthermore, users can start, stop, or adjust the pump remotely using their smartphone.

The massaging and suction system may be any of the systems disclosed herein, with the appropriate accessories/parts. During operation, the massaging and suction system may be placed on the user's breast. The suction and massage functions may be performed simultaneously or at different times, either dependently or independently.

At block 1120, the suction control settings may be configured, for example, through the UI provided by the massaging and suction system or a connected device/system.

At block 1122, based on the settings, suction from the vacuum pump is transmitted to the diaphragm through pneumatic components.

At block 1124, the diaphragm contracts and expands to create suction in the flange (e.g., the flange base 830 or another suitable flange, such as the flange 312) and the flange plug (e.g., the flange plug 810). The flange and the flange plug, when assembled, may be referred to as a flange-flange plug assembly.

At block 1126, suction is applied to the nipple through the flange-flange plug assembly which simulates milk expression. The flange-flange plug assembly according to an embodiment may include the flange plug 810 and the flange base 830 as depicted in FIG. 8A. The flange-flange plug assembly according to an embodiment may include the flange plug 810 and the flange 312 as depicted in FIG. 13. However, it should be noted that the flange-flange plug assembly may be formed by other suitable components.

At block 1140, the expressed milk passes through the duckbill valve (e.g., the duckbill valve 870) and is connected in a milk vessel (e.g., the collection vessel 860).

At block 1130, the massage control settings may be configured, for example, through the UI provided by the massaging and suction system or a connected device/system.

At block 1132, compressed air from the air pump travels through the pneumatic components to the “nozzle” of the massage cushions (or other suitable port/opening of the massage cushions).

At block 1134, a solenoid valve assembly (e.g., the solenoid valve) controls the direction of airflow and allows inflation and deflation of cushion chambers (or pods/bladders).

At block 1136, cyclic inflation and deflation of the massage cushions compress and massage the breast.

The massaging process (e.g., corresponding to blocks 1130, 1132, 1134, and/or 1136) may aid in the milk extraction process facilitated by blocks 1120, 1122, 1124, and/or 1126, thereby increasing the milk collected at block 1140 and/or enhancing the user's comfort during extraction/suction.

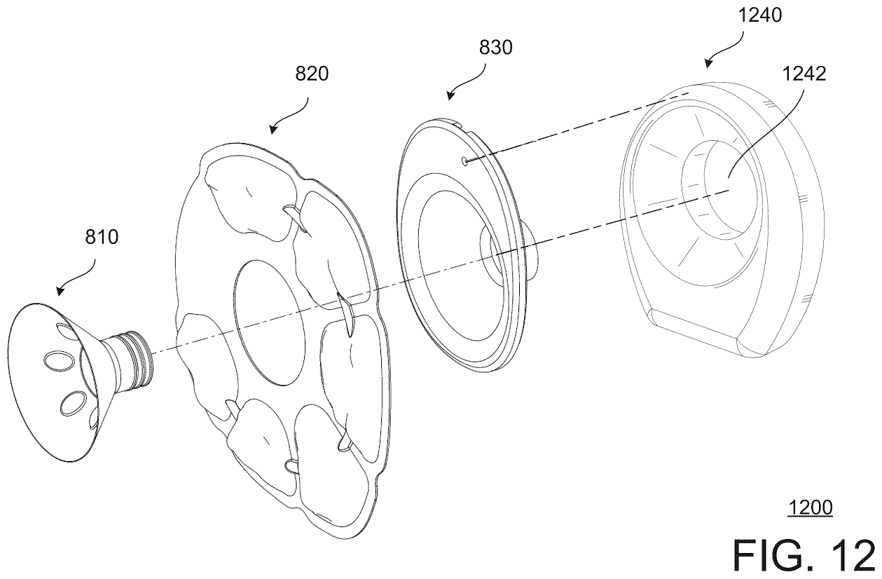

FIG. 12 illustrates an example massaging and suction system 1200, in accordance with some embodiments. The massaging and suction system 1200 includes the flange plug 810, the massage cushions 820, the flange base 830, a controller device 1240, and/or other suitable components. In this example, the controller device 1240 is designed with an opening 1242, into which the flange base 830 can be inserted. Similar to the other controller devices described herein, the controller device 1240 may include the massage assembly and/or the suction assembly. In certain embodiments, the massaging and suction system 1200 may operate in conjunction with other suitable components, such as another breast pumping system, to provide massaging and/or suction functions.

FIG. 13 illustrates an example massaging and suction system 1300, in accordance with some embodiments. The massaging and suction system 1300 includes the flange plug 810, the massage cushions 820, and a controller device 1340. The breast pump system 310 is described in detail with reference to FIG. 3A. The flange plug 810 can be plugged into the flange 312. The massage cushions 820 are positioned between the flange 312 and the flange plug 810, ensuring proper alignment. The controller device 1340 may include the massage assembly and/or the suction assembly. In this configuration, the controller device 1340 may be configured to provide massage functions, while the suction functions (e.g., milk extraction) are controlled by the control system 318 in the breast pump system 310.

The massaging and suction system 1300 may be utilized as an adaptable massage system, designed to function as an independent massage enhancement module compatible with any wearable or standard breast pump (e.g., the breast pump system 310). In certain embodiments, the massaging and suction system 1300 is solely dedicated to massaging the breast during pumping, ensuring enhanced comfort and improved milk expression while remaining universally adaptable to existing breast pump systems.

In certain embodiments, the controller device 1340 does not include a vacuum pump for suction; instead, the controller device 1340 focuses solely on regulating airflow for the inflation and deflation of the massage cushions 820. This design allows the massaging and suction system 1300 (e.g., the controller device 1340) to be small, lightweight, and ergonomic, ensuring case of use and portability.

In certain embodiments, the controller device 1340 is designed to snap directly into the massage cushions 820. This feature ensures that the entire system remains compact and wearable, without requiring external tubing or bulky attachments.

In certain embodiments, the flange plug 810 in this configuration acts as a universal adapter, allowing the massage cushions 820 to be used with any breast pump flange (e.g., the flange 312). The flange plug 810 may be constructed from medical-grade silicone or similar flexible, biocompatible materials, ensuring both durability and comfort. The massage cushions 820 work synergistically with the flange plug 810, ensuring that the compression cycles remain uniform and effective, promoting optimal milk flow and improved comfort.

In certain embodiments, the flange plug 810 may incorporate one or more of the following features. For example, a tapered, conical shape may be incorporated, ensuring a secure and airtight fit inside the flange (e.g., the flange 312) of a breast pump. Adaptive sizing options may be incorporated, allowing for different insert diameters to accommodate various pump models. Structural reinforcement may be incorporated, maintaining stability and preventing deformation under continuous compression cycles. Secure locking mechanism may be incorporated, preventing dislodgement during operation while remaining easy to remove for cleaning and repositioning.

By plugging directly into the flange of any pump, the flange plug 810 ensures that the massage cushions 820 remain securely positioned without interfering with the suction mechanism of the pump itself.

FIGS. 14A-14C illustrate example variants 1410-1490 of the massage cushions, in accordance with some embodiments. The massage cushions provide localized, adjustable compression, mimicking the stimulation provided by hand expression. As shown in FIGS. 14A-14C, varied chamber configurations may be implemented, including concentric, spiral, paired, interconnected pockets, or any combination thereof, allowing different inflation patterns that create unique sensations. The chambers may be connected through channels. The massage cushions may be integrated with one or more ports, such as nozzles or openings, to allow air (or other substance, such as liquid) to flow into or out of the massage cushions. The chambers of the massage cushions may be inflated sequentially, simultaneously, or in a hybrid manner, depending on the arrangement of the port(s) and/or the operation of the controller device (e.g., the massage assembly integrated therein).

As shown in FIG. 14A, when utilizing a single nozzle, the chambers in the corresponding cushions may inflate following the direction of the arrows. Specifically, the massage cushions 1440 may be inflated following the directions of arrows 1442, 1444, and then 1446.

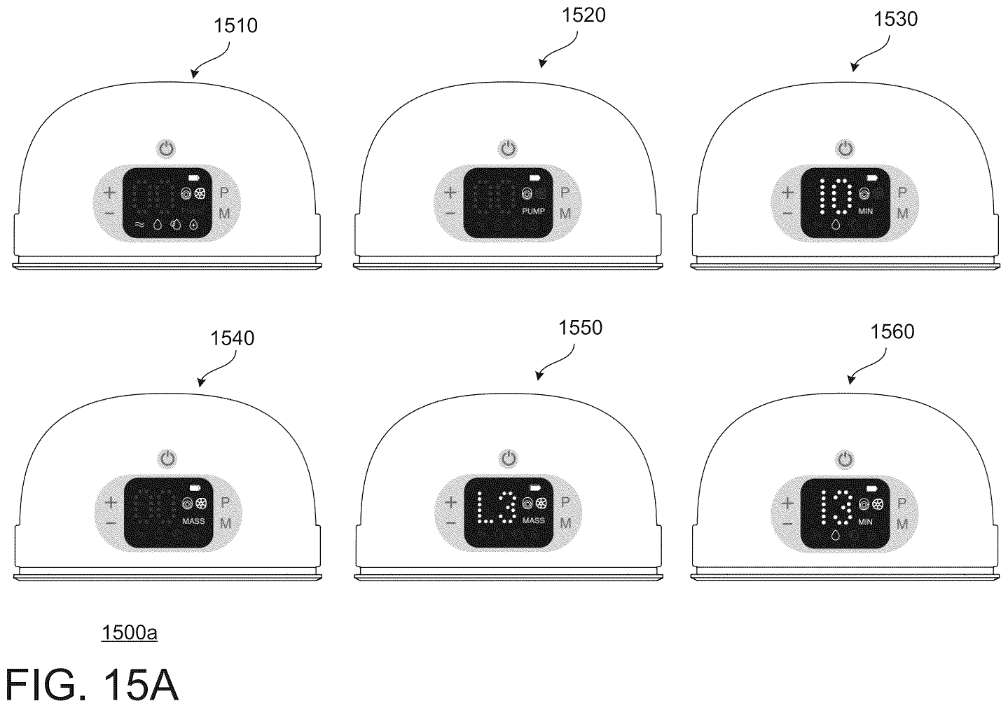

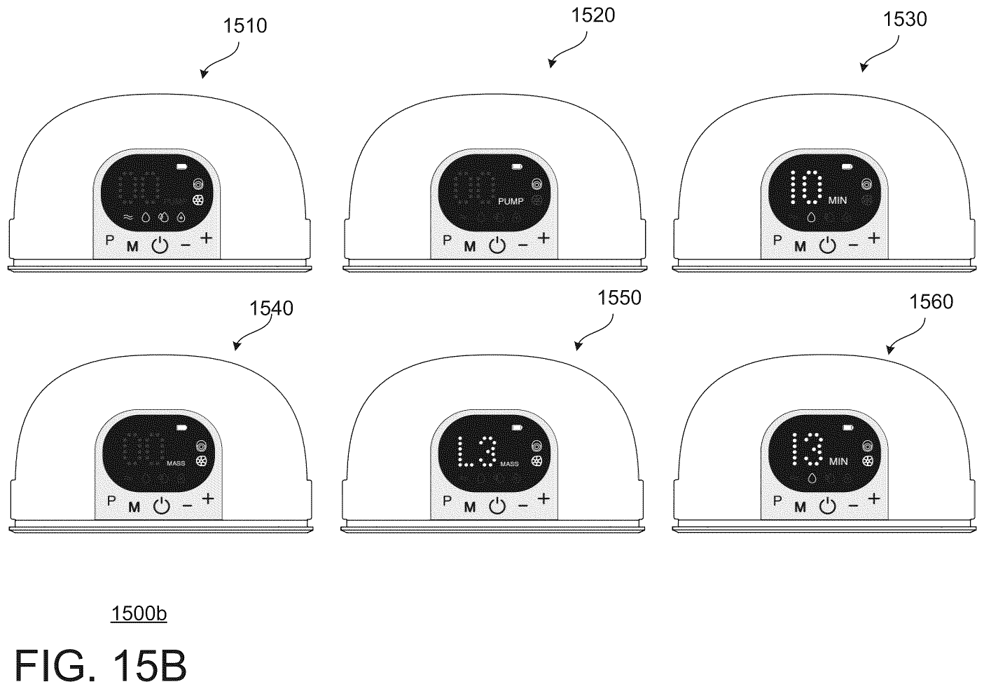

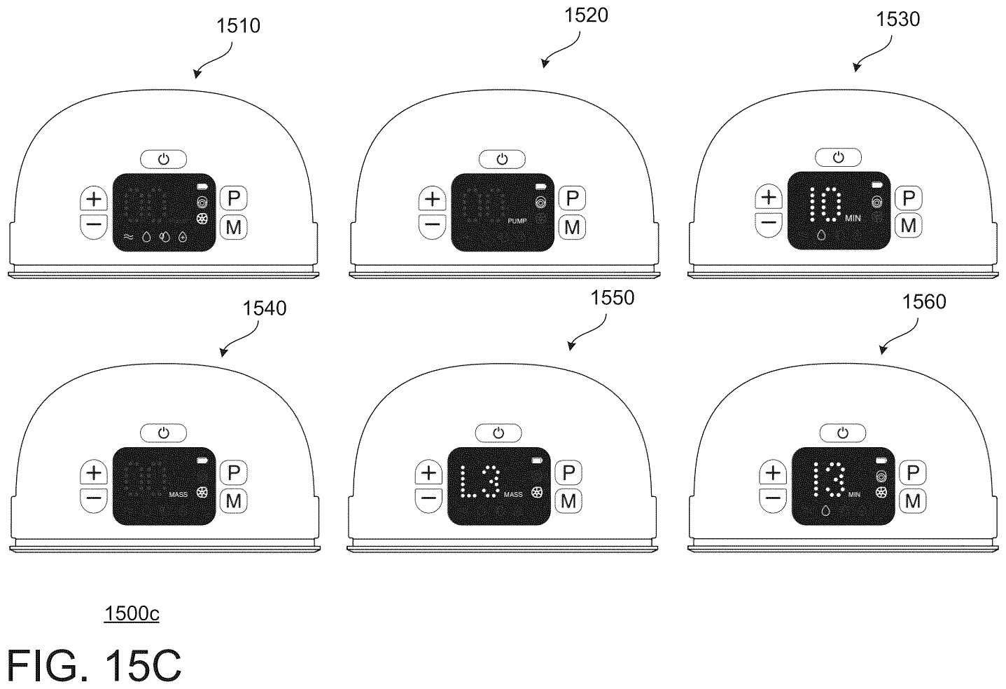

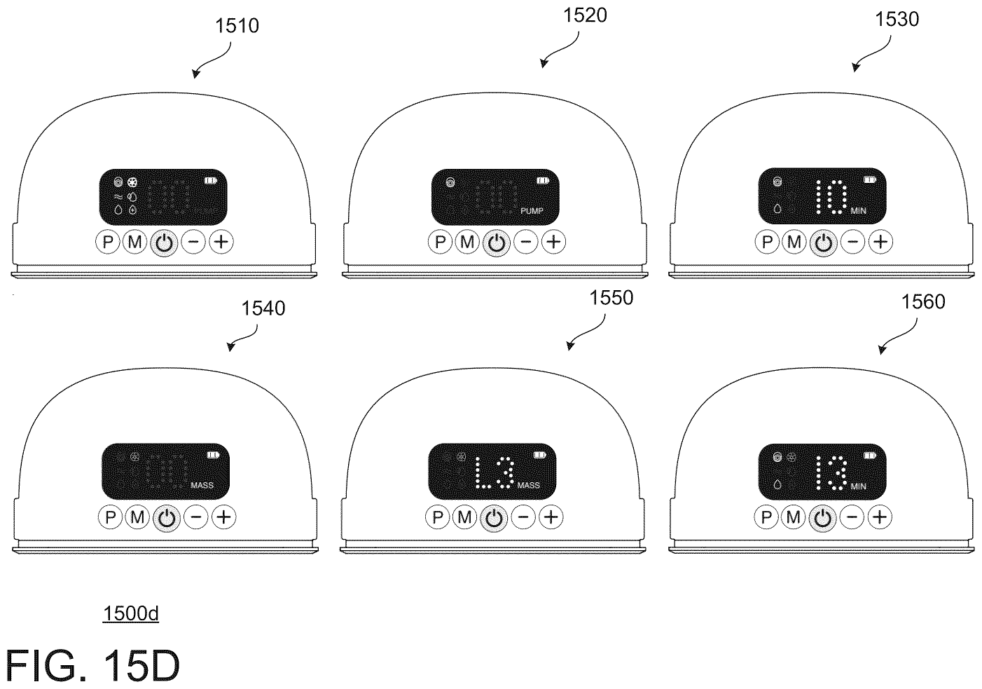

FIGS. 15A-15D illustrates examples of UIs 1500a-1500d, in accordance with some embodiments. The UI includes one or more buttons and a display interface, which may be integrated in a controller device in any of the massaging and suction systems provided in the present disclosure. FIGS. 145-15D shows the status of the UI at various stages 1510-1560.

At stage 1510, the power button is pressed. This operation causes all functions and modes to light up.

At stage 1520, the pump function automatically starts.

At stage 1530, the “P” button is pressed to toggle through modes. For example, an expression mode may be selected. The pumping time corresponding to the selected mode is displayed on the screen.

At stage 1540, the “M” button is pressed, which selects the massage function.

At stage 1550, the “+” or “−” button is pressed to increase or decrease the intensity level for the selected function (and/or the selected mode).

At stage 1560, the screen shows the in-progress status. For example, the display indicates that the pump function is on in the expression mode with the pumping session ends in 13 minutes, the massage function is activated, and the battery power status is full.

The massaging and suction systems provided in the present disclosure are not limited to breastfeeding applications but can also be utilized for broader breast health and therapeutic purposes. In certain embodiments, with minor modifications to the massage cushions, such as making the central hole optional when milk flow is not required, the massaging and suction systems can be adapted for various non-lactation uses. This flexibility allows for targeted compression therapy, enhancing the versatility of the systems beyond breastfeeding support.

One significant application is post-surgical breast compression therapy for lymphedema management. Following breast surgery, particularly in cases of mastectomy or lumpectomy, individuals often experience swelling due to fluid retention in the lymphatic system. The rhythmic inflation and deflation of the massage cushions can aid in lymphatic drainage, reducing swelling and discomfort while promoting circulation and tissue recovery.

Another potential use is in menopausal breast health maintenance. As individuals approach menopause, hormonal changes can lead to breast tenderness, reduced elasticity, and changes in tissue density. The massaging and suction systems can provide gentle compression therapy, improving circulation and helping maintain breast tissue flexibility. This can be particularly beneficial in reducing discomfort associated with hormonal fluctuations and supporting overall breast wellness.

The massaging and suction systems can also serve as an effective pain relief tool for conditions such as fibrocystic breasts, a common condition where the breast tissue becomes lumpy and tender due to hormonal influences. By using controlled compression cycles, the massage cushions can help alleviate discomfort, reduce fluid retention in the breast tissue, and enhance circulation, providing relief without the need for medications or invasive procedures.

The integration of smart features enhances the massaging and suction systems' utility beyond lactation. The ability to track milk production, analyze trends, and sync with mobile applications allows users to monitor their pumping efficiency and overall breast health. In the case of therapeutic applications, the massaging and suction systems' sensors can provide real-time health insights, track changes in breast tissue response, and offer customized compression settings based on user preferences or medical guidance. This data-driven approach not only optimizes lactation support but also facilitates preventative breast care and personalized therapy for a range of breast health concerns.

In certain embodiments, the massaging and suction systems may be modified and used for postpartum breast therapy, reducing engorgement and discomfort, lymphatic drainage applications, improving circulation and relieving breast swelling, and general breast massage therapy, aiding in relaxation and tissue health.

It is noted that the techniques described herein may be embodied in executable instructions stored in a computer readable medium for use by or in connection with a processor-based instruction execution machine, system, apparatus, or device. It will be appreciated by those skilled in the art that, for some embodiments, various types of computer-readable media can be included for storing data. As used herein, a “computer-readable medium” includes one or more of any suitable media for storing the executable instructions of a computer program such that the instruction execution machine, system, apparatus, or device may read (or fetch) the instructions from the computer-readable medium and execute the instructions for carrying out the described embodiments. Suitable storage formats include one or more of an electronic, magnetic, optical, and electromagnetic format. A non-exhaustive list of conventional exemplary computer-readable medium includes: a portable computer diskette; a random-access memory (RAM); a read-only memory (ROM); an erasable programmable read only memory (EPROM); a flash memory device; and optical storage devices, including a portable compact disc (CD), a portable digital video disc (DVD), and the like.

It should be understood that the arrangement of components illustrated in the attached Figures are for illustrative purposes and that other arrangements are possible. For example, one or more of the elements described herein may be realized, in whole or in part, as an electronic hardware component. The elements may be implemented in software, hardware, or a combination of software and hardware. Moreover, some or all of these other elements may be combined, some may be omitted altogether, and additional components may be added while still achieving the functionality described herein. Thus, the subject matter described herein may be embodied in many different variations, and all such variations are contemplated to be within the scope of the claims.

To facilitate an understanding of the subject matter described herein, many aspects are described in terms of sequences of actions. It will be recognized by those skilled in the art that the various actions may be performed by specialized circuits or circuitry, by program instructions being executed by one or more processors, or by a combination of both. The description herein of any sequence of actions is not intended to imply that the specific order described for performing that sequence must be followed. All methods described herein may be performed in any suitable order unless otherwise indicated herein or otherwise clearly contradicted by context.

The use of the terms “a” and “an” and “the” and similar references in the context of describing the subject matter (particularly in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. Furthermore, the foregoing description is for the purpose of illustration only, and not for the purpose of limitation, as the scope of protection sought is defined by the claims as set forth hereinafter together with any equivalents thereof. The use of any and all examples, or exemplary language (e.g., “such as”) provided herein, is intended merely to better illustrate the subject matter and does not pose a limitation on the scope of the subject matter unless otherwise claimed. The use of the term “based on” and other like phrases indicating a condition for bringing about a result, both in the claims and in the written description, is not intended to foreclose any other conditions that bring about that result. No language in the specification should be construed as indicating any non-claimed element as essential to the practice of the invention as claimed.

What is claimed is:

1. A massaging and suction system, comprising:
a cushion device comprising:
a plurality of chambers that are interconnected by one or more channels;
one or more outlets placed on the cushion device, wherein the plurality of chambers are inflated and deflated through the one or more outlets; and
a flange insert providing an interface for suction operations; and
a controller device comprising:
a first pump configured to inflate the plurality of chambers through the one or more outlets to facilitate massaging operations.

2. The massaging and suction system according to claim 1, wherein the cushion device is formed by sealing a flange layer and a cushion layer together,
wherein the flange layer comprises:
a rim,
a first opening, and
the one or more outlets connectable to the controller device,
wherein the cushion layer comprises:
a second opening,
a plurality of ridges arranged along the second opening, and
one or more channel-shaped structures, and
wherein the first opening of the flange layer aligns with the second opening of the cushion layer, the plurality of ridges in the cushion layer and the rim of the flange layer form the plurality of chambers in the interconnected cushion, the one or more channel-shaped structures and the rim of the flange layer form the one or more channels connecting adjacent chambers of the plurality of chambers, and the flange layer serves as the flange insert.

3. The massaging and suction system according to claim 1,
wherein the cushion device further comprises:
a flange plug having a conical shape; and
a cushion pad comprising the plurality of chambers, wherein the one or more outlets are situated on the cushion pad; and
wherein when assembled, the flange plug is inserted into a flange, and the cushion pad is placed between the flange plug and the flange, and
wherein the flange plug serves as the flange insert.

4. The massaging and suction system according to claim 3, wherein the cushion device further comprises a flange base, wherein the flange base serves as the flange, wherein the flange base comprises one or more openings, and the one or more outlets on the cushion pad are connectable to the controller device through the one or more openings on the flange base.

5. The massaging and suction system according to claim 1, wherein the plurality of chambers comprise one or more of:
concentric pockets;
spiral configurations;
paired pockets; or
a series of interconnected chambers.

6. The massaging and suction system according to claim 1, wherein the plurality of chambers are inflated simultaneously or sequentially.

7. The massaging and suction system according to claim 1, wherein the cushion device is integrated or assembled with the flange insert, wherein a separate suction device is configured to provide suction through the flange insert.

8. The massaging and suction system according to claim 7, wherein the controller device is configured to apply a massaging operation that is synchronized with a suction operation provided by the suction device.

9. The massaging and suction system according to claim 1, wherein the controller device is configured to adjust a massaging operation based on a suction operation.

10. The massaging and suction system according to claim 1, further comprising:
one or more sensors, wherein the controller device is configured to adjust at least one of a massaging operation or a suction operation based on data obtained from the one or more sensors.

11. The massaging and suction system according to claim 1, wherein the controller device further comprises:
a second pump configured to apply suction to facilitate suction operations.

12. The massaging and suction system according to claim 11, wherein the controller device is configured to operate massage and suction functions independently.

13. The massaging and suction system according to claim 11, wherein the controller device is configured to apply a massaging operation and a suction operation simultaneously.

14. A method for performing massage using a massaging and suction system, comprising:
inflating, by a controller device of the massaging and suction system, a plurality of chambers of a cushion device comprising:
the plurality of chambers that are interconnected by one or more channels; and
one or more outlets placed on the cushion device, wherein the plurality of chambers are inflated and deflated through the one or more outlets; and
a flange insert in contact with a user, wherein the flange insert provides an interface for suction operations; and
deflating, by the controller device of the massaging and suction system, the plurality of chambers.

15. The method according to claim 14, wherein the cushion device is formed by sealing a flange layer and a cushion layer together,
wherein the flange layer comprises:
a rim,
a first opening, and
the one or more outlets connectable to the controller device,
wherein the cushion layer comprises:
a second opening,
a plurality of ridges arranged along the second opening, and
one or more channel-shaped structures, and
wherein the first opening of the flange layer aligns with the second opening of the cushion layer, the plurality of ridges in the cushion layer and the rim of the flange layer form the plurality of chambers in the interconnected cushion, the one or more channel-shaped structures and the rim of the flange layer form the one or more channels connecting adjacent chambers of the plurality of chambers, and the flange layer serves as the flange insert.

16. The method according to claim 14,
wherein the cushion device further comprises:
a flange plug having a conical shape; and
a cushion pad comprising the plurality of chambers, wherein the one or more outlets are situated on the cushion pad; and
wherein when assembled, the flange plug is inserted into a flange, and the cushion pad is placed between the flange plug and the flange, and
wherein the flange plug serves as the flange insert.

17. The method according to claim 14, wherein the plurality of chambers are inflated simultaneously or sequentially.

18. The method according to claim 14, further comprising:
applying suction to the user through the flange insert.

19. The method according to claim 18, wherein the massaging operations and the suction are synchronized.

20. The method according to claim 18, wherein the suction is applied using a separate suction device, or
wherein the suction is applied using the controller device of the massaging and suction system.
